SEQUENCE LISTING (11 sequences)

SEQ ID NO: 1 - protein, 25 aa, Homo sapiens
HDRHAGTTSDVSSYGAAKAWVKGRG

SEQ ID NO: 2 - protein, 34 aa, Homo sapiens
HADGSFSDEMNTILDNLAARDFINWLIQTKITDR

SEQ ID NO: 3 - protein, 29 aa, Homo sapiens
HSQGTFTSDYSKYLDSRRAQDFVQWLMNT

SEQ ID NO: 4 - protein, 27 aa, Homo sapiens
HSDGLFTSEYSKMRGNAQVQKFIQNLM

SEQ ID NO: 5 - protein, 27 aa, Homo sapiens
HADGVFTSDYSRLLGQLSAKKYLESLI

SEQ ID NO: 6 - protein, 42 aa, Homo sapiens
YAEGTFISDYSIAMDKIHQQDFVNWLLAQKGKKNDWKHNITQ

SEQ ID NO: 7 - protein, 38 aa, Homo sapiens
HSDGIFTDSYSRYRKQMAVKKYLAAVLGKRYKQRVKNK

SEQ ID NO: 8 - protein, 101 aa, Homo sapiens
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN

SEQ ID NO: 9 - protein, 115 aa, Homo sapiens
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 10 - protein, 115 aa, Homo sapiens
MVSRKAVAALLVVHVAAMLASQTEAFVPIFTYGELQRMQEKERNKGQKKSLSVWQRSGEEGPVDPAEPIREEENEMIKLTAPLEIGMRMNSRQLEKYPATLEGLLSEMLPQHAAK

SEQ ID NO: 11 - protein, 95 aa, Homo sapiens
MAAARLCLSLLLLSTCVALLLQPLLGAQGAPLEPVYPGDNATPEQMAQYAADLRRYINMLTRPRYGKRHKEDTLAFSEWGSPHAAVPRELSPLDL

(19) United States
(12) Patent Application Publication
(10) Pub. No.: US 2008/0311138 A1
(43) Pub. Date: Dec. 18, 2008

(54) Adjuvant Activity of Gastrointestinal Peptides

(76) Inventor: Maria Teresa De Magistris, Rome (IT)

Correspondence Address: BANNER & WITCOFF, LTD., 1100 13th STREET, N.W., SUITE 1200, WASHINGTON, DC, 20005-4051, US

(21) Appl. No.: 11/792,947

(22) PCT Filed: Dec. 13, 2005
(86) PCT No.: PCT/IB05/04088

§ 371 Date: Jun. 11, 2008

(30) Foreign Application Priority Data
Dec. 13, 2004 (GB) 0427267.0

(51) Int. Cl. A61K 39/00; A61P 37/02

(52) U.S. Cl. 424/184.1

(57) ABSTRACT

Gastrointestinal peptides (GPs) have been found to function as vaccine adjuvants, and in particular as mucosal adjuvants. The invention provides an immunogenic composition comprising: (a) a GP adjuvant; and (b) an antigen. The composition is preferably suitable for mucosal administration e.g. intranasal administration.

All documents cited herein are incorporated by reference in their entirety.

### TECHNICAL FIELD

This invention is in the field of vaccine adjuvants, particularly mucosal vaccine adjuvants.

### BACKGROUND ART

Vaccination with purified antigens alone is typically insufficient to elicit a protective immune response, so vaccines almost always require formulation with adjuvants. Different adjuvants have different immunological profiles, which can match the deferent requirements of different vaccines, and there is an ongoing need for new adjuvants for inclusion in vaccines.

While current vaccines primarily target the systemic immune system, many pathogens are transmitted across the mucosal surfaces of the body, initiating either localised infection (e.g. rotavirus, the parainfluenza viruses and respiratory syncytial virus) or disseminating from the mucosa to systemic tissues (e.g. HIV, measles and Mycobacteria tuberculosis). Vaccines that can prevent pathogen dissemination from the initial site(s) of infection are a priori likely to be more successful than those that target the blood or disease stage tissue or organs. Moreover, successful mucosal vaccination would obviate the need for injections and facilitate self-administration in some cases. However, the development of vaccines directed to the mucosal surfaces of the body has been stymied by the lack of adjuvants licensed for use at mucosal surfaces.

Aluminium salts are the only universally licensed adjuvant for human use. However, these induce a Th2 response and are not suitable for mucosal immunisation.

Weakness or absence of responsiveness to mucosal vaccination in the absence of an effective mucosal adjuvant still remains the major concern for prophylactic or therapeutic vaccines. Effective mucosal adjuvants are known in the art e.g. CpG oligonucleotides, LT mutants and chitosan. However, there remains a need for further effective mucosal adjuvants.

### DISCLOSURE OF THE INVENTION

Surprisingly, it has been found that gastrointestinal peptides (GP) can function as vaccine adjuvants, and in particular as mucosal adjuvants, when administered in combination with an antigen. Therefore, the invention provides an immunogenic composition comprising: (a) a gastrointestinal peptide adjuvant; and (b) at least one antigen.

The composition is preferably suitable for mucosal administration e.g. intranasal administration.

The term ‘gastrointestinal peptide’ (GP) refers to hormones or neuropeptides that are secreted by enteroendocrine cells and by gastrointestinal nerves. The adjuvant included in the compositions of the invention may be any suitable gastrointestinal peptide, or agonist thereof, including those which are isolated from a naturally occurring source and those which are chemically synthesised.

Preferably, the GP can induce cAMP production in epithelial cells. This effect can conveniently be measured in vitro by contacting the GP with live epithelial cells and checking for an increase in cAMP levels. Kits for cAMP measurement are readily available from commercial suppliers.

Preferred GP adjuvants can bind to G protein coupled receptors, in particular to the Gi and/or Gs and/or Gq protein coupled receptors. GPs that bind to Gs proteins are particularly preferred.

The GP may be selected from the group consisting of VIP (Vasoactive Intestinal Peptide), PACAP (Pituitary Adenylate Cyclase-Activating Polypeptide), gastrin, a cholecystokinin, motilin, neurotensin, secretin, glucagon, miniglucagon, GIP (Gastric Intestinal Peptide; also known as Glucose-dependent Insulinotropic Polypeptide), enteroglucagon, pancreatic polypeptide (PP), glicentin, glicentin-related pancreatic peptide (GRPP), oxyntomodulin, GLP-1 (Glucagon-Like Peptide 1) and GLP-2 (Glucagon-Like Peptide 2). GLP-1 and GIP are known as incretins. Incretins are preferred GPs for use as adjuvants, and GLP-1 is particularly preferred.

GRPP, glucagon, GLP-1, GLP-2, glicentin and oxyntomodulin are proteolytic cleavage products of the same polypeptide, as shown in FIG. 3.

Gastrointestinal Peptides

GLP-1 (e.g. GenBank accession No. CAA24759; SEQ ID NO:1) is a peptide that is produced by post-translational processing of preproglucagon. The biological activities of GLP-1 include stimulation of glucose-dependent insulin secretion and insulin biosynthesis, inhibition of glucagon secretion and gastric emptying, and inhibition of food intake. GLP-1 appears to have various additional effects in the GI tract and central nervous system [1,2].

GLP-1 is synthesised in intestinal endocrine cells in 4 forms: 1-37, 7-37, 1-36 amide and 7-36 amide. The ‘amide’ forms of GLP-1 are amidated at the C-terminus. The full length N-terminal extended forms of GLP-1 (1-37 and 1-36 amide) are generally devoid of biological activity. Removal of the first 6 amino acids results in a shorter version of the GLP-1 (7-36 amide) molecule with substantially enhanced biological activity. The majority of circulating biologically active GLP-1 is found in the ‘7-36 amide’ form, with lesser amounts of the bioactive ‘7-37’ form also detectable [3]. Both of these peptides appear equivalent in all biological features studied to date. Following synthesis, the levels of the bioactive forms of these peptides fall rapidly, largely due to renal clearance and the N-terminal degradation of both peptides by dipeptidyl peptidase IV (‘DPP-IV’). This widely expressed enzyme cleaves GLP-1 at Ala-2, resulting in the generation of inactive GLP-1^{9-36 amide} and GLP-1⁹⁻³⁷, respectively. The expression of DPP-IV in the gut and vascular endothelium is consistent with findings that the majority of immunoreactive GLP-1 entering the portal venous circulation has already been inactivated by N-terminal cleavage, accounting for its short half life (several minutes).

Any of the GLP-1 variants known in the art may be used with the invention, but the ‘7-36 amide’ form is preferred.

GLP-2 (e.g. GenBank accession CAA24759; SEQ ID NO:2) is a 33 amino acid peptide, co-secreted along with GLP-1 from intestinal endocrine cells in the small and large intestine. The biological activities of GLP-2 include stimulation of mucosal growth in the small and large intestine, inhibition of enterocyte and crypt cell apoptosis, stimulation of enterocyte glucose transport and GLUT-2 expression and inhibition of gastric emptying and gastric acid secretion. GLP-2 also has actions outside the GI tract, including stimulation of cell proliferation in rat astrocyte cell cultures [4].

GLP-2, like GLP-1, is subject to N-terminal degradation by the enzyme DPP-IV. Accordingly, GLP-2 analogues that are resistant to DPP-IV are more potent in vivo [5]. Following cleavage of full length extended GLP-2 (1-33) by DPP-IV, bioinactive GLP-2 (3-33) is liberated.

Glucagon (e.g. GenBank accession No. CAA24759; SEQ ID NO:3) is a 29 amino acid peptide hormone liberated in the a cells of the islets of Langerhans. Glucagon opposes the action of insulin in peripheral tissues, predominantly the liver, where the insulin:glucagon ratio determines the control of gluconeogenesis and glycogenolysis. The tissue-specific liberation of proglucagon is controlled by cell-specific expression of prohormone convertase (PC) enzymes. Glucagon is also synthesized in the CNS, where its actions may include regulation of peripheral glucoregulation, yet remain less well understood.

A proteolytic fragment of 29 amino acid glucagon (19-29), a ‘miniglucagon’, is liberated following cleavage of glucagon at Arg17-Arg18 [6]. Processing may occur locally in target tissues such as the pancreas, liver or heart, as well as in the circulation. To date, a separate receptor for miniglucagon has not been identified, although various actions have been ascribed to this peptide, including effects in the liver, heart and pancreas, including inhibition of insulin secretion.

Glicentin, oxyntomodulin and GRPP are further cleavage products of the preproglucagon precursor (FIG. 3). Enteroglucagon is similar to glicentin, and originates from the terminal ileum and the colon. It delays gastric emptying and has trophic effects on gut mucosa.

Secretin (e.g. GenBank accession No. P01280; SEQ ID NO:4) is a 27 amino acid basic peptide produced by S cells and released by acid delivered into the duodenum. Secretin is released into the blood when duodenal pH drops below 4. Secretin is a potent stimulus for bicarbonate secretion and also stimulates secretion of bile, release of insulin, and release of gastric pepsin in the stomach. Secretin inhibits glucagon release, intestinal motility, and prevents the uptake of water and sodium ions by the intestine.

VIP (e.g. GenBank accession No. P81401; SEQ ID NO:5) is a 28-amino acid peptide structurally related to secretin. It was originally isolated from intestinal extracts and shown to be a potent vasodilator. VIP is very widely distributed in the peripheral and central nervous systems. VIP 1-28 induces smooth muscle relaxation (lower esophageal sphincter, stomach, gallbladder), stimulates secretion of water into pancreatic juice and bile, and causes inhibition of gastric acid secretion and absorption from the intestinal lumen. The VIP 2-28 gastrointestinal peptide behaves as a full VIP agonist in man and demonstrates similar biological activity to the parent peptide [7].

GIP (e.g. GenBank accession No. P09681; SEQ ID NO:6) is a 42 amino acid peptide hormone synthesized in and secreted from K cells in the intestinal epithelium. GIP secretion is primarily regulated by nutrients, especially fat. The primary action of GIP is the stimulation of glucose-dependent insulin secretion. An important determinant of GIP action is the N-terminal cleavage of the full length extended bioactive 1-48 peptide to the inactive GIP (3-42). The enzyme DPP-IV, which also cleaves GLP-1 and GLP-2, rapidly inactivates GIP both in vivo.

PACAP (e.g. GenBank accession No. P41535; SEQ ID NO: 7 herein) elicits various biological actions as a neurotransmitter and neuromodulator via three heptahelical G-protein-linked receptors. PACAP also acts as an insulinotropic factor. PACAP exists in two amidated forms, PACAP 1-38 (SEQ ID NO:7) and PACAP 1-27 sharing the same N-terminal 27 amino acids, which are alternatively processed forms of a 176-amino acid precursor (pre-proPACAP).

Gastrin is synthesized as a 101 residue pre-pro-peptide (e.g. GenBank accession No. AAH69762; SEQ ID NO:8 herein) and is post-translationally modified by cleavage and α-amidation to result in the active forms G34, G17 and G13/14 (‘big’, ‘little’ and ‘mini’ gastrins). Sulfation of an internal tyrosine residue distinguishes type I gastrins from type II (sulfated) gastrins.

Cholecystokinins are the cleavage products of the procholecystokinin precursor (e.g. GenBank accession No. NP_{—}000720; SEQ ID NO:9 herein). The linear peptide is synthesized as a preprohormone, then proteolytically cleaved to generate a family of peptides having the same C-termini. Full biologic activity is retained in CCK-8 (8 amino acids), but peptides of 33, 38 and 59 amino acids are also produced.

Motilin (e.g. GenBank accession No. NP_{—}002409; SEQ ID NO:10 herein) is a 22 amino acid peptide secreted by endocrinocytes in the mucosa of the proximal small intestine.

Pancreatic polypeptide (e.g. GenBank accession No. NP_{—}002713; SEQ ID NO:11 herein) is a 36 amino acid secretory peptide that is predominantly produced by the pancreas.

Agonists

Peptides that behave as GP agonists are also suitable for use in the invention. The term “agonist” refers to a substance that has affinity for and stimulates physiological activity at a cell receptor normally stimulated by naturally occurring substances, thus triggering a biochemical response. For example, the agonist may stimulate the GP receptor. In relation to GLP-1, an example of a suitable GLP-1 agonist is exendin-4, a peptide of 39 amino acids isolated from the venom of the Gila monster lizard, which has 53% sequence homology to GLP-1. Exendin-4 is a full agonist at the GLP-1 receptor [8]. In relation to PACAP, an example of a suitable agonist is Maxadilan, a potent vasodilator peptide isolated from salivary glands extracts of the hematophagous sand fly [9].

Gastrointestinal peptides have a number of advantages when used as mucosal adjuvants. They are endogenous molecules and are therefore devoid of general toxicity. The peptides may be conserved among species and have a short half life in the circulation e.g. GLP-1 which, after mucosal administration, is unlikely to reach pancreatic beta cells to stimulate insulin secretion. GLP-1 is a small peptide which is economic to produce synthetically in a form devoid of LPS contamination. Furthermore, as the peptides are stable upon temperature change, their use as mucosal adjuvants enables the production and transport of temperature-stable vaccines. This property is particularly desirable for vaccines used in developing countries.

The GP adjuvant may have an amino acid sequence having at least c% sequence identity to the amino acid sequence of a native GP (e.g. SEQ ID NOs: 1-9) and/or comprising an amino acid sequence consisting of a fragment of at least x contiguous amino acids from an amino acid sequence of a native GP. This includes GP variants (e.g. allelic variants, homologs, orthologs, paralogs, mutants, etc.). For example, variants of GLP-1 that have been disclosed in the prior art include a form in which Ala-8 is replaced by Ser, thereby preventing breakdown by DPP IV.

The value of c is at least 75 e.g. 75, 80, 85, 86, 87, 88, 89, 90, 91, 92, 93, 94, 95, 96, 97, 98, 99, 99.5, or more. The value of x is at least 5 e.g. 6, 7, 8, 9, 10, 11, 12, 13, 14, 15, 16, 17, 18, 19, 20, 21, 22, 23, 24, 25, 26, 27, 28, 29, 30. The skilled person can generate variants and fragments of a native GP and can screen them for adjuvant activity e.g. using the experimental methods disclosed in the examples. For example, known fragments of GLP-1 include the ‘9-36’ form that arises from the action of DPP IV. The natural N-terminal two amino acids (His-Ala-) are cleaved by DPP IV, and the shortened GLP-1 molecule loses biological activity.

Peptides as short as 5-mers have been shown to induce cytokine production from cells in vitro [10]. Although the over-riding factor that determines the length of the peptide is that it has to possess adjuvant activity, other factors may also contribute to the determination of the final length of the peptide. Such factors may include the expense involved in manufacturing said polypeptide, with shorter polypeptides being cheaper to synthesise.

Preferably, the GP sequence is derived from the mammalian species in which it will be used as an adjuvant. For example, when the compositions of the present invention are to be used in human patients, the GP sequence is derived from the human GP.

The GP adjuvant can be formulated in various ways within the composition.

The GP is preferably present in a soluble aqueous form. If the composition is formulated as an oil-in-water emulsion then the adjuvant will usually be present in the aqueous phase.

The GP will generally be used at a concentration of between 1 and 500 μg/dose.

Antigens

The compositions of the invention are preferably immunogenic e.g. vaccines. Vaccines according to the invention may either be prophylactic (i.e. to prevent infection) or therapeutic (i.e. to treat disease after infection), but will typically be prophylactic. The compositions may be used to treat or prevent infections caused by any of the below-listed pathogens.

The compositions of the invention therefore comprise an immunologically effective amount of at least one antigen. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Antigens suitable for use in the compositions of the invention may be bacterial or viral antigens. Suitable antigens may be further classified as protein antigens, carbohydrate antigens or glycoconjugate antigens. The compositions of the invention may include one or more antigens. Antigens for use with the invention include, but are not limited to, one or more of the following antigens set forth below, or antigens derived from one or more of the pathogens set forth below.

Also useful are other antigens, compositions, methods, and microbes included in refs. 11 to 15, which are contemplated in conjunction with the compositions of the present invention.

The following references include antigens useful in conjunction with the compositions of the present invention:

A. Bacterial Antigens

Bacterial antigens suitable for use in the invention include proteins, polysaccharides, lipopolysaccharides, and outer membrane vesicles which may be isolated, purified or derived from a bacteria. In addition, bacterial antigens may include bacterial lysates and inactivated bacteria formulations. Bacteria antigens may be produced by recombinant expression. Bacterial antigens preferably include epitopes which are exposed on the surface of the bacteria during at least one stage of its life cycle. Bacterial antigens are preferably conserved across multiple serotypes. Bacterial antigens include antigens derived from one or more of the bacteria set forth below as well as the specific antigens examples identified below.

Neisseria meningitidis: meningococcal antigens may include proteins (such as those identified in references 16-22], saccharides (including a polysaccharide, oligosaccharide or lipopolysaccharide), or outer-membrane vesicles [23-26] purified or derived from a N. meningitidis serogroup such as A, C, W135, Y, and/or B. Meningococcal protein antigens may be selected from adhesins, autotransporters, toxins, iron acquisition proteins, and membrane associated proteins (preferably integral outer membrane proteins). See also refs. 27-35.

Streptococcus pneumoniae: S. pneumoniae antigens may include a saccharide (including a polysaccharide or an oligosaccharide) and/or protein from S. pneumoniae. Saccharide antigens may be selected from serotypes 1, 2, 3, 4, 5, 6B, 7F, 8, 9N, 9V, 10A, 11A, 12F, 14, 15B, 17F, 18C, 19A, 19F, 20, 22F, 23F, and 33F. Protein antigens may be selected, for example, from a protein identified in any of refs. 36-41. S. pneumoniae proteins may be selected from the Poly Histidine Triad family (PhtX), the Choline Binding Protein family (CbpX), CbpX truncates, LytX family, LytX truncates, CbpX truncate-LytX truncate chimeric proteins, pneumolysin (Ply), PspA, PsaA, Sp128, Sp101, Sp130, Sp125 or Sp133. See also refs. 42-48.

Streptococcus pyogenes (Group A Streptococcus): Group A Streptococcus antigens may include a protein identified in reference 49 or 50 (including GAS40), fusions of fragments of GAS M proteins (including those described in refs. 51-53, fibronectin binding protein (Sfb1), Streptococcal heme-associated protein (Shp), and Streptolysin S (SagA). See also refs. 49, 54 and 55.

Moraxella catarrhalis: Moraxella antigens include antigens identified in refs. 56 & 57, outer membrane protein antigens (HMW-OMP), C-antigen, and/or LPS. See also ref. 58.

Bordetella pertussis: Pertussis antigens include pertussis holotoxin (PT) and filamentous haemagglutinin (FHA) from B. pertussis, optionally also combination with pertactin and/or agglutinogens 2 and 3 antigen. See also refs. 59 & 60.

Staphylococcus aureus: S. aureus antigens include S. aureus type 5 and 8 capsular polysaccharides optionally conjugated to nontoxic recombinant Pseudomonas aeruginosa exotoxin A, such as StaphVAX™, or antigens derived from surface proteins, invasins (leukocidin, kinases, hyaluronidase), surface factors that inhibit phagocytic engulfment (capsule, Protein A), carotenoids, catalase production, Protein A, coagulase, clotting factor, and/or membrane-damaging toxins (optionally detoxified) that lyse eukaryotic cell membranes (hemolysins, leukotoxin, leukocidin). See also ref. 61.

Staphylococcus epidermis: S. epidermidis antigens include slime-associated antigen (SAA).

Clostridium tetani (Tetanus): Tetanus antigens include tetanus toxoid (TT), preferably used as a carrier protein in conjunction/conjugated with the compositions of the present invention.

Corynebacterium diphtheriae (Diphtheria): Diphtheria antigens include diphtheria toxin or detoxified mutants thereof, such as CRM197. Additionally antigens capable of modulating, inhibiting or associated with ADP ribosylation are contemplated for combination/co-administration/conjugation with the compositions of the present invention. These diphtheria antigens may be used as carrier proteins.

Haemophilus influenzae: H. influenzae antigens include a saccharide antigen from type B, or protein D [62].

Pseudomonas aeruginosa: Pseudomonas antigens include endotoxin A, Wzz protein, P. aeruginosa LPS, more particularly LPS isolated from PAO1 (O5 serotype), and/or Outer Membrane Proteins, including Outer Membrane Proteins F (OprF) [63].

Legionella pneumophila. Bacterial antigens may be derived from Legionella pneumophila.

Streptococcus agalactiae (Group B Streptococcus): Group B Streptococcus antigens include a protein or saccharide antigen identified in refs. 49 and 64-67. For example, the antigens include proteins GBS80, GBS104, GBS276 and GBS322, and/or saccharide antigens derived from serotypes Ia, Ib, Ia/c, II, III, IV, V, VI, VII and VIII).

Neisseria gonorrhoeae: Gonococcal antigens include Por (or porin) protein, such as PorB [68], a transferring binding protein, such as TbpA and TbpB [69], a opacity protein (such as Opa), a reduction-modifiable protein (Rmp), and outer membrane vesicle (OMV) preparations [70]. See also refs. 16, 17, 18 & 71.

Chlamydia trachomatis: C. trachomatis antigens include antigens derived from serotypes A, B, Ba and C (agents of trachoma, a cause of blindness), serotypes L₁, L₂ & L₃ (associated with Lymphogranuloma venereum), and serotypes, D-K. C. trachomatis antigens may also include an antigen identified in refs. 67 & 72-74, including PepA (CT045), LcrE (CT089), ArtJ (CT381), DnaK (CT396), CT398, OmpH-like (CT242), L7/L12 (CT316), OmcA (CT444), AtosS (CT467), CT547, Eno (CT587), HrtA (CT823), and MurG (CT761). See also ref. 75.

Treponema pallidum (Syphilis): Syphilis antigens include TmpA antigen.

Haemophilus ducreyi (causing chancroid): Ducreyi antigens include outer membrane protein (DsrA).

Enterococcus faecalis or Enterococcus faecium: Antigens include a trisaccharide repeat or other Enterococcus derived antigens provided in ref. 76.

Helicobacter pylori: H. pylori antigens include Cag, Vac, Nap, HopX, HopY and/or urease antigen. [77-87].

Staphylococcus saprophyticus: Antigens include the 160 kDa hemagglutinin of S. saprophyticus antigen.

Yersinia enterocolitica Antigens include LPS [88].

Escherichia coli: E. coli antigens may be derived from enterotoxigenic E. coli (ETEC), enteroaggregative E. coli (EAggEC), diffusely adhering E. coli (DAEC), enteropathogenic E. coli (EPEC), and/or enterohemorrhagic E. coli (EHEC) strains.

Bacillus anthracis (anthrax): B. anthracis antigens are optionally detoxified and may be selected from A-components (lethal factor (LF) and edema factor (EF)), both of which can share a common B-component known as protective antigen (PA). See refs. 89-91.

Yersinia pestis (plague): Plague antigens include F1 capsular antigen [92], LPS [93],V antigen [94].

Mycobacterium tuberculosis: Tuberculosis antigens include lipoproteins, LPS, BCG antigens, a fusion protein of antigen 85B (Ag85B) and/or ESAT-6 optionally formulated in cationic lipid vesicles [95], Mycobacterium tuberculosis (Mtb) isocitrate dehydrogenase associated antigens [96], and/or MPT51 antigens [97].

Rickettsia: Antigens include outer membrane proteins, including the outer membrane protein A and/or B (OmpB) [98], LPS, and surface protein antigen (SPA) [99].

Listeria monocytogenes: Bacterial antigens may be derived from Listeria monocytogenes.

Chlamydia pneumoniae: Antigens include those identified in refs. 72 & 100 to 105.

Vibrio cholerae: Antigens include proteinase antigens, LPS, particularly lipopolysaccharides of Vibrio cholerae II, O1 Inaba O-specific polysaccharides, V. cholera O139, antigens of IEM108 vaccine [106], and/or Zonula occludens toxin (Zot).

Salmonella typhi (typhoid fever): Antigens include capsular polysaccharides preferably conjugates (Vi, e.g. vax-TyVi).

Borrelia burgdorferi (Lyme disease): Antigens include lipoproteins (such as OspA, OspB, Osp C and Osp D), other surface proteins such as OspE-related proteins (Erps), decorin-binding proteins (such as DbpA), and antigenically variable VI proteins. , such as antigens associated with P39 and P13 (an integral membrane protein, [107], VlsE Antigenic Variation Protein [108].

Porphyromonas gingivalis: Antigens include the outer membrane protein (OMP). See also ref. 109.

Klebsiella: Antigens include an OMP, including OMP A, or a polysaccharide optionally conjugated to tetanus toxoid.

Further bacterial antigens of the invention may be capsular antigens, polysaccharide antigens or protein antigens of any of the above. Further bacterial antigens may also include an outer membrane vesicle (OMV) preparation. Additionally, antigens include live, attenuated, and/or purified versions of any of the aforementioned bacteria. The antigens of the present invention may be derived from gram-negative or gram-positive bacteria. The antigens of the present invention may be derived from aerobic or anaerobic bacteria.

Additionally, any of the above bacterial-derived saccharides (polysaccharides, LPS, LOS or oligosaccharides) can be conjugated to another agent or antigen, such as a carrier protein (for example CRM197). Such conjugation may be direct conjugation effected by reductive amination of carbonyl moieties on the saccharide to amino groups on the protein, as provided in refs. 110 & 111. Alternatively, the saccharides can be conjugated through a linker, such as, with succinimide or other linkages provided in refs. 166 & 168.

B. Viral Antigens

Viral antigens suitable for use in the invention include inactivated (or killed) virus, attenuated virus, split virus formulations, purified subunit formulations, viral proteins which may be isolated, purified or derived from a virus, and Virus Like Particles (VLPs). Viral antigens may be derived from viruses propagated on cell culture or other substrate. Alternatively, viral antigens may be expressed recombinantly. Viral antigens preferably include epitopes which are exposed on the surface of the virus during at least one stage of its life cycle. Viral antigens are preferably conserved across multiple serotypes or isolates. Viral antigens include antigens derived from one or more of the viruses set forth below as well as the specific antigens examples identified below.

Orthomyxovirus: Viral antigens may be derived from an Orthomyxovirus, such as Influenza A, B and C. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase components (PB1, PB2 and PA). Preferred antigens include HA and NA.

Influenza antigens may be derived from interpandemic (annual) flu strains. Alternatively influenza antigens may be derived from strains with the potential to cause pandemic a pandemic outbreak (i.e., influenza strains with new haemagglutinin compared to the haemagglutinin in currently circulating strains, or influenza strains which are pathogenic in avian subjects and have the potential to be transmitted horizontally in the human population, or influenza strains which are pathogenic to humans).

Paramyxoviridae viruses: Viral antigens may be derived from Paramyxoviridae viruses, such as Pneumoviruses (RSV), Paramyxoviruses (PIV) and Morbilliviruses (Measles). [112-114].

Pneumovirus: Viral antigens may be derived from a Pneumovirus, such as Respiratory syncytial virus (RSV), Bovine respiratory syncytial virus, Pneumonia virus of mice, and Turkey rhinotracheitis virus. Preferably, the Pneumovirus is RSV. Pneumovirus antigens may be selected from one or more of the following proteins, including surface proteins Fusion (F), Glycoprotein (G) and Small Hydrophobic protein (SH), matrix proteins M and M2, nucleocapsid proteins N, P and L and nonstructural proteins NS1 and NS2. Preferred Pneumovirus antigens include F, G and M. See, for example, ref. 115. Pneumovirus antigens may also be formulated in or derived from chimeric viruses. For example, chimeric RSV/PIV viruses may comprise components of both RSV and PIV.

Paramyxovirus: Viral antigens may be derived from a Paramyxovirus, such as Parainfluenza virus types 1-4 (PIV), Mumps, Sendai viruses, Simian virus 5, Bovine parainfluenza virus and Newcastle disease virus. Preferably, the Paramyxovirus is PIV or Mumps. Paramyxovirus antigens may be selected from one or more of the following proteins: Hemagglutinin-Neuraminidase (HN), Fusion proteins F1 and F2, Nucleoprotein (NP), Phosphoprotein (P), Large protein (L), and Matrix protein (M). Preferred Paramyxovirus proteins include HN, F1 and F2. Paramyxovirus antigens may also be formulated in or derived from chimeric viruses. For example, chimeric RSV/PIV viruses may comprise components of both RSV and PIV. Commercially available mumps vaccines include live attenuated mumps virus, in either a monovalent form or in combination with measles and rubella vaccines (MMR).

Morbillivirus: Viral antigens may be derived from a Morbillivirus, such as Measles. Morbillivirus antigens may be selected from one or more of the following proteins: hemagglutinin (H), Glycoprotein (G), Fusion factor (F), Large protein (L), Nucleoprotein (NP), Polymerase phosphoprotein (P), and Matrix (M). Commercially available measles vaccines include live attenuated measles virus, typically in combination with mumps and rubella (MMR).

Picornavirus: Viral antigens may be derived from Picornaviruses, such as Enteroviruses, Rhinoviruses, Heparnavirus, Cardioviruses and Aphthoviruses. Antigens derived from Enteroviruses, such as Poliovirus are preferred. See refs. 116 & 117.

Enterovirus: Viral antigens may be derived from an Enterovirus, such as Poliovirus types 1, 2 or 3, Coxsackie A virus types 1 to 22 and 24, Coxsackie B virus types 1 to 6, Echovirus (ECHO) virus) types 1 to 9, 11 to 27 and 29 to 34 and Enterovirus 68 to 71. Preferably, the Enterovirus is poliovirus. Enterovirus antigens are preferably selected from one or more of the following Capsid proteins VP1, VP2, VP3 and VP4. Commercially available polio vaccines include Inactivated Polio Vaccine (IPV) and oral poliovirus vaccine (OPV).

Heparnavirus: Viral antigens may be derived from an Heparnavirus, such as Hepatitis A virus (HAV). Commercially available HAV vaccines include inactivated HAV vaccine. [118,119].

Togavinus: Viral antigens may be derived from a Togavirus, such as a Rubivirus, an Alphavirus, or an Arterivirus. Antigens derived from Rubivirus, such as Rubella virus, are preferred. Togavirus antigens may be selected from E1, E2, E3, C, NSP-1, NSPO-2, NSP-3 or NSP-4. Togavirus antigens are preferably selected from E1, E2 or E3. Commercially available Rubella vaccines include a live cold-adapted virus, typically in combination with mumps and measles vaccines (MMR).

Flavivirus: Viral antigens may be derived from a Flavivirus, such as Tick-borne encephalitis (TBE), Dengue (types 1, 2, 3 or 4), Yellow Fever, Japanese encephalitis, West Nile encephalitis, St. Louis encephalitis, Russian spring-summer encephalitis, Powassan encephalitis. Flavivirus antigens may be selected from PrM, M, C, E, NS-1, NS-2a, NS2b, NS3, NS4a, NS4b, and NS5. Flavivirus antigens are preferably selected from PrM, M and E. Commercially available TBE vaccine include inactivated virus vaccines.

Pestivirus: Viral antigens may be derived from a Pestivirus, such as Bovine viral diarrhea (BVDV), Classical swine fever (CSFV) or Border disease (BDV).

Hepadnavirus: Viral antigens may be derived from a Hepadnavirus, such as Hepatitis B virus. Hepadnavirus antigens may be selected from surface antigens (L, M and S), core antigens (HBc, HBe). Commercially available HBV vaccines include subunit vaccines comprising the surface antigen S protein. [119,120].

Hepatitis C virus: Viral antigens may be derived from a Hepatitis C virus (HCV). HCV antigens may be selected from one or more of E1, E2, E1/E2, NS345 polyprotein, NS 345-core polyprotein, core, and/or peptides from the nonstructural regions [121,122].

Rhabdovirus: Viral antigens may be derived from a Rhabdovirus, such as a Lyssavirus (Rabies virus) and Vesiculovirus (VSV). Rhabdovirus antigens may be selected from glycoprotein (G), nucleoprotein (N), large protein (L), nonstructural proteins (NS). Commercially available Rabies virus vaccine comprise killed virus grown on human diploid cells or fetal rhesus lung cells. [123,124].

Caliciviridae; Viral antigens may be derived from Calciviridae, such as Norwalk virus, and Norwalk-like Viruses, such as Hawaii Virus and Snow Mountain Virus.

Coronavirus: Viral antigens may be derived from a Coronavirus, SARS, Human respiratory coronavirus, Avian infectious bronchitis (IBV), Mouse hepatitis virus (MHV), and Porcine transmissible gastroenteritis virus (TGEV). Coronavirus antigens may be selected from spike (S), envelope (E), matrix (M), nucleocapsid (N), and Hemagglutinin-esterase glycoprotein (HE). Preferably, the Coronavirus antigen is derived from a SARS virus. SARS viral antigens are described in ref. 125.

Retrovirus: Viral antigens may be derived from a Retrovirus, such as an Oncovirus, a Lentivirus or a Spumavirus. Oncovirus antigens may be derived from HTLV-1, HTLV-2 or HTLV-5. Lentivirus antigens may be derived from HIV-1 or HIV-2. Retrovirus antigens may be selected from gag, pol, env, tax, tat, rex, rev, nef, vif, vpu, and vpr. HIV antigens may be selected from gag (p24gag and p55gag), env (gp160, gp120 and gp41), pol, tat, nef, rev vpu, miniproteins, (preferably p55 gag and gp140v delete). HIV antigens may be derived from one or more of the following strains: HIV_{IIIb}, HIV_{SF2}, HIV_{LAV}, HIV_{LAI}, HIV_{MN}, HIV-1_{CM235}, HIV-1_{US4}.

Reovirus: Viral antigens may be derived from a Reovirus, such as an Orthoreovirus, a Rotavirus, an Orbivirus, or a Coltivirus. Reovirus antigens may be selected from structural proteins λ1, λ2, λ3, μ1, μ2, σ1, σ2, or σ3, or nonstructural proteins σNS, μNS, or σ1s. Preferred Reovirus antigens may be derived from a Rotavirus. Rotavirus antigens may be selected from VP1, VP2, VP3, VP4 (or the cleaved product VP5 and VP8), NSP 1, VP6, NSP3, NSP2, VP7, NSP4, or NSP5. Preferred Rotavirus antigens include VP4 (or the cleaved product VP5 and VP8), and VP7.

Parvovirus: Viral antigens may be derived from a Parvovirus, such as Parvovirus B19. Parvovirus antigens may be selected from VP-1, VP-2, VP-3, NS-1 and NS-2. Preferably, the Parvovirus antigen is capsid protein VP-2.

Delta hepatitis virus (HDV): Viral antigens may be derived HDV, particularly δ-antigen from HDV (see, e.g., ref. 126).

Hepatitis E virus (HEV): Viral antigens may be derived from HEV.

Hepatitis G virus (HGV): Viral antigens may be derived from HGV.

Human Herpesvirus: Viral antigens may be derived from a Human Herpesvirus, such as Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8). Human Herpesvirus antigens may be selected from immediate early proteins (α), early proteins (β), and late proteins (γ). HSV antigens may be derived from HSV-1 or HSV-2 strains. HSV antigens may be selected from glycoproteins gB, gC, gD and gH, fusion protein (gB), or immune escape proteins (gC, gE, or gI). VZV antigens may be selected from core, nucleocapsid, tegument, or envelope proteins. A live attenuated VZV vaccine is commercially available. EBV antigens may be selected from early antigen (EA) proteins, viral capsid antigen (VCA), and glycoproteins of the membrane antigen (MA). CMV antigens may be selected from capsid proteins, envelope glycoproteins (such as gB and gH), and tegument proteins

Papovaviruses: Antigens may be derived from Papovaviruses, such as Papillomaviruses and Polyomaviruses. Papillomaviruses include HPV serotypes 1, 2, 4, 5, 6, 8, 11, 13, 16, 18, 31, 33, 35, 39, 41, 42, 47, 51, 57, 58, 63 and 65. Preferably, HPV antigens are derived from serotypes 6, 11, 16 or 18. HPV antigens may be selected from capsid proteins (L1) and (L2), or E1-E7, or fusions thereof. HPV antigens are preferably formulated into virus-like particles (VLPs). Polyomyavirus viruses include BK virus and JK virus. Polyomavirus antigens may be selected from VP1, VP2 or VP3.

C. Fungal Antigens

Fungal antigens may be derived from one or more of the fungi set forth below.

Fungal antigens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme.

Fungal pathogens include Aspergillus fumigatus, Aspergillus flavus, Aspergillus niger, Aspergillus nidulans, Aspergillus terreus, Aspergillus sydowi, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Paracoccidioides brasiliensis, Pneumocystis carinii, Pythiumn insidiosum, Pityrosporum ovale, Saccharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

Processes for producing a fungal antigens are well known in the art [127]. In a preferred method a solubilized fraction extracted and separated from an insoluble fraction obtainable from fungal cells of which cell wall has been substantially removed or at least partially removed, characterized in that the process comprises the steps of: obtaining living fungal cells; obtaining fungal cells of which cell wall has been substantially removed or at least partially removed; bursting the fungal cells of which cell wall has been substantially removed or at least partially removed; obtaining an insoluble fraction; and extracting and separating a solubilized fraction from the insoluble fraction.

D. STD Antigens

The compositions of the invention may include one or more antigens derived from a sexually transmitted disease (STD). Such antigens may provide for prophylactis or therapy for STD's such as chlamydia, genital herpes, hepatitis (such as HCV), genital warts, gonorrhoea, syphilis and/or chancroid [128]. Antigens may be derived from one or more viral or bacterial STD's. Viral STD antigens for use in the invention may be derived from, for example, HIV, herpes simplex virus (HSV-1 and HSV-2), human papillomavirus (HPV), and hepatitis (HCV). Bacterial STD antigens for use in the invention may be derived from, for example, Neisseria gonorrhoeae, Chlamydia trachomatis, Treponema pallidum, Haemophilus ducreyi, Escherichia coli, and Streptococcus agalactiae. Examples of specific antigens derived from these pathogens are described above.

E. Respiratory Antigens

The compositions of the invention may include one or more antigens derived from a pathogen which causes respiratory disease. For example, respiratory antigens may be derived from a respiratory virus such as Orthomyxoviruses (influenza), Pneumovirus (RSV), Paramyxovirus (PIV), Morbillivirus (measles), Togavirus (Rubella), VZV, and Coronavirus (SARS). Respiratory antigens may be derived from a bacteria which causes respiratory disease, such as Streptococcus pneumoniae, Pseudomonas aeruginosa, Bordetella pertussis, Mycobacterium tuberculosis, Mycoplasma pneumoniae, Chlamydia pneumoniae, Bacillus anthracis, and Moraxella catarrhalis. Examples of specific antigens derived from these pathogens are described above.

F. Pediatric Vaccine Antigens

The compositions of the invention may include one or more antigens suitable for use in pediatric subjects. Pediatric subjects are typically less than about 3 years old, or less than about 2 years old, or less than about 1 years old. Pediatric antigens may be administered multiple times over the course of 6 months, 1, 2 or 3 years. Pediatric antigens may be derived from a virus which may target pediatric populations and/or a virus from which pediatric populations are susceptible to infection. Pediatric viral antigens include antigens derived from one or more of Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), and Varicella-zoster virus (VZV), Epstein Barr virus (EBV). Pediatric bacterial antigens include antigens derived from one or more of Streptococcus pneumoniae, Neisseria meningitidis, Streptococcus pyogenes (Group A Streptococcus), Moraxella catarrhalis, Bordetella pertussis, Staphylococcus aureus, Clostridium tetani (Tetanus), Corynebacterium diphtheriae (Diphtheria), Haemophilus influenzae type B (Hib), Pseudomonas aeruginosa, Streptococcus agalactiae (Group B Streptococcus), and Escherichia coli. Examples of specific antigens derived from these pathogens are described above.

G. Antigens Suitable for Use in Elderly or Immunocompromised Individuals

The compositions of the invention may include one or more antigens suitable for use in elderly or immunocompromised individuals. Such individuals may need to be vaccinated more frequently, with higher doses or with adjuvanted formulations to improve their immune response to the targeted antigens. Antigens which may be targeted for use in Elderly or Immunocompromised individuals include antigens derived from one or more of the following pathogens: Neisseria meningitidis, Streptococcus pneumoniae, Streptococcus pyogenes (Group A Streptococcus), Moraxella catarrhalis, Bordetella pertussis, Staphylococcus aureus, Staphylococcus epidermis, Clostridium tetani (Tetanus), Corynebacterium diphtheriae (Diphtheria), Haemophilus influenzae type B (Hib), Pseudomonas aeruginosa, Legionella pneumophila, Streptococcus agalactiae (Group B Streptococcus), Enterococcus faecalis, Helicobacter pylori, Chlamydia pneumoniae, Orthomyxovirus (influenza), Pneumovirus (RSV), Paramyxovirus (PIV and Mumps), Morbillivirus (measles), Togavirus (Rubella), Enterovirus (polio), HBV, Coronavirus (SARS), Varicella-zoster virus (VZV), Epstein Barr virus (EBV), Cytomegalovirus (CMV). Examples of specific antigens derived from these pathogens are described above.

H. Antigens Suitable for Use in Adolescent Vaccines

The compositions of the invention may include one or more antigens suitable for use in adolescent subjects. Adolescents may be in need of a boost of a previously administered pediatric antigen. Pediatric antigens which may be suitable for use in adolescents are described above. In addition, adolescents may be targeted to receive antigens derived from an STD pathogen in order to ensure protective or therapeutic immunity before the beginning of sexual activity. STD antigens which may be suitable for use in adolescents are described above.

I. Tumor Antigens

One embodiment of the invention involves a tumor antigen or cancer antigen. Tumor antigens can be, for example, peptide-containing tumor antigens, such as a polypeptide tumor antigen or glycoprotein tumor antigens. A tumor antigen can also be, for example, a saccharide-containing tumor antigen, such as a glycolipid tumor antigen or a ganglioside tumor antigen. The tumor antigen can further be, for example, a polynucleotide-containing tumor antigen that expresses a polypeptide-containing tumor antigen, for instance, an RNA vector construct or a DNA vector construct, such as plasmid DNA.

Tumor antigens appropriate for the practice of the present invention encompass a wide variety of molecules, such as (a) polypeptide-containing tumor antigens, including polypeptides (which can range, for example, from 8-20 amino acids in length, although lengths outside this range are also common), lipopolypeptides and glycoproteins, (b) saccharide-containing tumor antigens, including poly-saccharides, mucins, gangliosides, glycolipids and glycoproteins, and (c) polynucleotides that express antigenic polypeptides.

The tumor antigens can be, for example, (a) full length molecules associated with cancer cells, (b) homologs and modified forms of the same, including molecules with deleted, added and/or substituted portions, and (c) fragments of the same. Tumor antigens can be provided in recombinant form. Tumor antigens include, for example, class I-restricted antigens recognized by CD8+ lymphocytes or class II-restricted antigens recognized by CD4+ lymphocytes.

Numerous tumor antigens are known in the art, including: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT, (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSH-P1, PSM-P1, PSM-P2, associated with e.g., prostate cancer, (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Le^{x} (associated with, e.g., breast and colorectal cancer) as well as various mucins; glycoproteins may be coupled to a carrier protein (e.g., MUC-1 may be coupled to KLH); (ii) lipopolypeptides (e.g., MUC-1 linked to a lipid moiety); (iii) polysaccharides (e.g., Globo H synthetic hexasaccharide), which may be coupled to a carrier proteins (e.g., to KLH), (iv) gangliosides such as GM2, GM12, GD2, GD3 (associated with, e.g., brain, lung cancer, melanoma), which also may be coupled to carrier proteins (e.g., KLH).

Additional tumor antigens which are known in the art include p15, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like. These as well as other cellular components are described for example in reference 129 and references cited therein.

Polynucleotide-containing antigens in accordance with the present invention typically comprise polynucleotides that encode polypeptide cancer antigens such as those listed above. Preferred polynucleotide-containing antigens include DNA or RNA vector constructs, such as plasmid vectors (e.g., pCMV), which are capable of expressing polypeptide cancer antigens in vivo.

Tumor antigens may be derived, for example, from mutated or altered cellular components. After alteration, the cellular components no longer perform their regulatory functions, and hence the cell may experience uncontrolled growth. Representative examples of altered cellular components include ras, p53, Rb, altered protein encoded by the Wilms' tumor gene, ubiquitin, mucin, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor. These as well as other cellular components are described for example in ref. 130 and references cited therein.

Additionally, bacterial and viral antigens, may be used in conjunction with the compositions of the present invention for the treatment of cancer. In particular, carrier proteins, such as CRM197, tetanus toxoid, or Salmonella typhimurium antigen can be used in conjunction/conjugation with compounds of the present invention for treatment of cancer. The cancer antigen combination therapies will show increased efficacy and bioavailability as compared with existing therapies.

Additional information on cancer or tumor antigens can be found, for example, in reference 131 (e.g. Tables 3 & 4), in reference 132 (e.g. Table 1) and in references 133 to 155.

Immunisation can also be used against Alzheimer's disease e.g. using Abeta as an antigen [156].

J. Antigen Formulations

In other aspects of the invention, methods of producing microparticles having adsorbed antigens are provided. The methods comprise: (a) providing an emulsion by dispersing a mixture comprising (i) water, (ii) a detergent, (iii) an organic solvent, and (iv) a biodegradable polymer selected from the group consisting of a poly(α-hydroxy acid), a polyhydroxy butyric acid, a polycaprolactone, a polyorthoester, a polyanhydride, and a polycyanoacrylate. The polymer is typically present in the mixture at a concentration of about 1% to about 30% relative to the organic solvent, while the detergent is typically present in the mixture at a weight-to-weight detergent-to-polymer ratio of from about 0.00001:1 to about 0.1:1 (more typically about 0.0001:1 to about 0.1:1, about 0.001:1 to about 0.1:1, or about 0.005:1 to about 0.1:1); (b) removing the organic solvent from the emulsion; and (c) adsorbing an antigen on the surface of the microparticles. In certain embodiments, the biodegradable polymer is present at a concentration of about 3% to about 10% relative to the organic solvent.

Microparticles for use herein will be formed from materials that are sterilizable, non-toxic and biodegradable. Such materials include, without limitation, poly(α-hydroxy acid), polyhydroxybutyric acid, polycaprolactone, polyorthoester, polyanhydride, PACA, and polycyanoacrylate. Preferably, microparticles for use with the present invention are derived from a poly(α-hydroxy acid), in particular, from a poly(lactide) (“PLA”) or a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered macromolecule. These parameters are discussed more fully below.

Additional formulation methods and antigens (especially tumor antigens) are provided in ref. 157.

Where a saccharide antigen is used, it is preferably conjugated to a carrier in order to enhance immunogenicity. Conjugation to carrier proteins is particularly useful for paediatric vaccines [e.g. ref. 158] and is a well known technique [e.g. reviewed in refs. 159 to 168, etc.], particularly for H. influenzae B, meningococcal and pneumococcal saccharide antigens. Saccharide antigens are thus preferably in the form of conjugates. Preferred carrier proteins for conjugates are bacterial toxins or toxoids, such as diphtheria toxoid or tetanus toxoid. The CRM197 mutant of diphtheria toxin [169-171] is a particularly preferred carrier for, as is a diphtheria toxoid. Other suitable carrier proteins include the N. meningitidis outer membrane protein [172], synthetic peptides [173, 174], heat shock proteins [175,176], pertussis proteins [177,178], cytokines [179], lymphokines [179], hormones [179], growth factors [179], artificial proteins comprising multiple human CD4⁺ T cell epitopes from various pathogen-derived antigens [180] such as the Ni 9 protein [181], protein D from H. influenzae [62,182], pneumococcal surface protein PspA [183], pneumolysin [184], iron-uptake proteins [185], toxin A or B from C. difficile [186], etc.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Toxic protein antigens may be detoxified where necessary (e.g. detoxification of pertussis toxin by chemical and/or genetic means).

Antigens in the composition will typically be present at a concentration of at least 1 μg/ml each. In general, the concentration of any given antigen will be sufficient to elicit an immune response against that antigen.

As an alternative to using proteins antigens in the mixture, nucleic acid encoding the antigen may be used. Protein components of the mixture may thus be replaced by nucleic acid (preferably DNA e.g. in the form of a plasmid) that encodes the protein. Similarly, compositions of the invention may comprise proteins which mimic saccharide antigens e.g. mimotopes [187] or anti-idiotype antibodies.

Fusion Proteins

The GP adjuvant and the antigen may be present as separate entities within the immunogenic composition. Alternatively, the GP adjuvant and the antigen may be associated with each other for example, the GP may be associated with the antigen through non-covalent bonds. Preferably, the GP may be fused to the antigen by covalent bonds, for example, through a peptide bond, through chemical linkage and so on. When the antigen is a protein antigen then the GP adjuvant and the antigen of the invention may be present as a fusion protein that can be translated as a single polypeptide.

There are a number of ways in which the GP may be fused with the antigen. For example, the GP may be fused to the antigen post-translationally e.g. by intein biology. Preferably, the GP is expressed as a genetic fusion, forming a single recombinant fusion protein with the antigen. In cases of such genetic fusions, the attachment of the GP and the antigen components may preferably be achieved using a recombinant DNA construct that encodes the amino acid sequence of the fusion protein, with the DNA encoding the GP in the same reading frame as the DNA encoding the antigen.

The GP may be fused to the amino or carboxy termini of the protein antigen, either directly or via a linker peptide e.g. a glycine rich oligopeptide. In this way, the GP may also be fused between two protein antigens through simultaneous fusion to the amino terminus of a first protein antigen and to the carboxy terminus of a second protein antigen, wherein the first and second antigens are the same or, preferably, different.

For expression in host cells, nucleic acid sequences encoding the fusion protein should be cloned into a suitable vector or vectors. The host cells may be transformed, transfected or transduced with such vectors and then cultured to achieve expression of the fusion protein. Suitable expression methods are well known to those of skill in the art and many are described in detail in references 188 & 189.

Pharmaceutical Compositions

The composition of the invention will typically, in addition to the components mentioned above, comprise one or more ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. A thorough discussion of pharmaceutically acceptable excipients is available in reference 190.

Compositions of the invention are generally presented in aqueous form (e.g. solutions or suspensions). In some embodiments of the invention the compositions are in aqueous form from the packaging stage to the administration stage (“full liquid vaccine”). In this way the composition can be administered direct from their packaged form, without the need for reconstitution in an aqueous medium. In other embodiments, however, one or more components of the compositions may be packaged in a lyophilised form, and a vaccine for actual administration may be reconstituted when necessary. Thus compositions of the invention may be prepared at a packaging stage, or may be prepared extemporaneously prior to use.

Compositions may be presented in vials or in ready-filled syringes. The syringes may be supplied with or without needles. A syringe will include a single dose of the composition, whereas a vial may include a single dose or multiple doses. However, preferred compositions are for mucosal delivery. Of the various mucosal delivery options available, the intranasal route may be the most practical as it offers easy access with relatively simple devices that have already been mass produced. The composition of the invention is thus preferably adapted for and/or packaged for intranasal administration, such as by nasal spray, nasal drops, gel or powder e.g. see refs. 191 & 192.

Alternative routes for mucosal delivery of the composition are oral, sublingual, intragastric, pulmonary, intestinal, transdermal, ocular and vaginal routes. The composition of the invention may thus be adapted for and/or packaged for mucosal administration e.g. refs. 193-195. Where the composition is for oral administration, for instance, it may be in the form of tablets or capsules (optionally enteric coated), liquid, transgenic plant material, drops, inhaler, aerosol, enteric coating, suppository, pessary etc. See also ref. 196 and chapter 17 of ref. 197.

Compositions of the invention may be packaged in unit dose form or in multiple dose form. For multiple dose forms, vials are preferred to pre-filled syringes. Effective dosage volumes can be routinely established, but a typical human dose of a composition for injection has a volume of about 0.5 ml. Similar doses may be used for other delivery routes e.g. an intranasal vaccine for atomisation may have a volume of about 125 μl per spray, with four sprays administered to give a total dose of about 0.5 ml.

The pH of the composition (including lyophilised compositions, after reconstitution) is preferably between 6 and 8, preferably about 7. Stable pH may be maintained by the use of a buffer. The composition may be sterile and/or pyrogen-free. Compositions of the invention may be isotonic with respect to humans.

Compositions of the invention may include an antimicrobial and/or a preservative, particularly when packaged in multiple dose format.

Compositions of the invention may comprise detergent e.g. a Tween (polysorbate), such as Tween 80. Detergents are generally present at low levels e.g. <0.01%.

Compositions of the invention may include sodium salts (e.g. sodium chloride) to give tonicity.

Compositions of the invention will generally include a buffer. A phosphate or histidine buffer is typical.

Methods of Treatment

The invention also provides a method for raising an immune response against at least one antigen, comprising the step of administering at least one antigen to a patient in combination with a GP adjuvant.

The invention also provides the use of: (a) a GP adjuvant; and (b) at least one antigen, in the manufacture of a medicament for administration to a patient to induce an immune response.

The GP adjuvant and the antigen(s) may be administered simultaneously, sequentially or separately. For example, the GP adjuvant may be administered to prime the patient before administration of the antigen(s) or after the administration of the antigen(s) to boost the patient's immune response to that antigen. The adjuvant and antigen(s) are preferably administered in admixture.

The invention also provides the use of at least one antigen in the manufacture of a medicament for raising an immune response in a patient, wherein the medicament is administered with a GP adjuvant. Similarly, the invention provides the use of a GP adjuvant in the manufacture of a medicament for raising an immune response in a patient, wherein the medicament is administered with at least one antigen.

The invention also provides the use of at least one antigen in the manufacture of a medicament for raising an immune response in a patient, where the patient has been pre-treated with a GP adjuvant. The invention also provides the use of a GP adjuvant in the manufacture of a medicament for raising an immune response in a patient, where the patient has been pre-treated with at least one antigen.

The invention also provides a composition of the invention for use in medicine.

A “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious pocket such as, for example, hamsters, chinchillas, ferrets, rats, guinea pigs, gerbils, rabbits and sugar gliders.

The patient is preferably a mammal, more preferably a human. Where the vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc.

The invention may be used to elicit systemic and/or mucosal immunity. For example, the invention may be used to elicit the production of specific IgA, IgG and/or IgM antibodies. The invention may also be used to elicit cell mediated immunity by promoting activation of antigen-specific CD4+ and/or CD8+ T lymphocytes.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

Preparing Immunogenic Compositions

The GP adjuvant of the invention is particularly suited to inclusion in immunogenic compositions and vaccines. A process of the invention may therefore include the step of mixing the adjuvant with an antigen. The invention provides a composition or vaccine obtainable in this way. Where a composition of the invention includes antigens from more than one organism, the antigens are preferably prepared separately and then admixed with the GP adjuvant to give a composition of the invention.

A composition of the invention may thus be prepared from a kit comprising: (a) a GP adjuvant and (b) at least one antigen. The GP and/or the at least one antigen may be present in lyophilised form. The invention also provides a method for preparing a composition of the invention, comprising mixing a GP adjuvant with one or more antigens (e.g. 1, 2, 3), wherein said one or more antigens are in liquid form.

Compositions of the invention may be formed by adding antigen to bulk adjuvant, or adding adjuvant to bulk antigen. Where the composition includes more than one antigen and/or more than one adjuvant, antigen(s) and adjuvant(s) may be mixed in any suitable order.

Further Adjuvants

GPs can act as adjuvants within the compositions of the invention. It is also possible to include one or more further adjuvants. Such adjuvants include, but are not limited to:

A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc. [e.g. see chapters 8 & 9 of ref. 197], or mixtures of different mineral compounds, with the compounds taking any suitable form (e.g. gel, crystalline, amorphous, etc.), and with adsorption being preferred. The mineral containing compositions may also be formulated as a particle of metal salt [198].

A typical aluminium phosphate adjuvant is amorphous aluminium hydroxyphosphate with PO₄/Al molar ratio between 0.84 and 0.92, included at 0.6mg Al³⁺/ml. Adsorption with a low dose of aluminium phosphate may be used e.g. between 50 and 100 μg Al³⁺ per conjugate per dose. Where an aluminium phosphate it used and it is desired not to adsorb an antigen to the adjuvant, this is favoured by including free phosphate ions in solution (e.g. by the use of a phosphate buffer).

B. Oil Emulsions

Oil emulsion compositions suitable for use as adjuvants in the invention include oil-in-water emulsions and water-in-oil emulsions.

A submicron oil-in-water emulsion may include squalene, Tween 80, and Span 85 e.g. with a composition by volume of about 5% squalene, about 0.5% polysorbate 80 and about 0.5% Span 85 (in weight terms, 4.3% squalene, 0.5% polysorbate 80 and 0.48% Span 85), known as ‘MF59’ [199-201 chapter 10 of ref. 197; chapter 12 of ref. 202]. The MF59 emulsion advantageously includes citrate ions e.g. 10 mM sodium citrate buffer.

An emulsion of squalene, a tocopherol, and Tween 80 can be used. The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably ≦1 as this provides a more stable emulsion. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.

An emulsion of squalene, a tocopherol, and a Triton detergent (e.g. Triton X-100) can be used.

An emulsion of squalane, polysorbate 80 and poloxamer 401 (“Pluronic™ L121”) can be used. The emulsion can be formulated in phosphate buffered saline, pH 7.4. This emulsion is a useful delivery vehicle for muramyl dipeptides, and has been used with threonyl-MDP in the “SAF-1” adjuvant [203] (0.05-1% Thr-MDP, 5% squalane, 2.5% Pluronic L121 and 0.2% polysorbate 80). It can also be used without the Thr-MDP, as in the “AF” adjuvant [204] (5% squalane, 1.25% Pluronic L121 and 0.2% polysorbate 80). Microfluidisation is preferred.

Complete Freund's adjuvant (CFA) and incomplete Freund's adjuvant (IFA) may also be used.

C. Saponin Formulations [Chapter 22 of Ref 197]

Saponin formulations may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 205. Saponin formulations may also comprise a sterol, such as cholesterol [206].

Combinations of saponins and cholesterols can be used to form unique particles called immunostimulating complexes (ISCOMs) [chapter 23 of ref. 197]. ISCOMs typically also include a phospholipid such as phosphatidylethanolamine or phosphatidylcholine. Any known saponin can be used in ISCOMs. Preferably, the ISCOM includes one or more of QuilA, QHA and QHC. ISCOMs are further described in refs. 206, 207 & 208]. Optionally, the ISCOMS may be devoid of additional detergent [209].

A review of the development of saponin based adjuvants can be found in refs. 210 & 211.

D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid-proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in [212]-[217]. Virosomes are discussed further in, for example [218].

E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 219. Such “small particles” of 3dMPL are small enough to be sterile filtered through a 0.22 μm membrane [219]. Other non-toxic LPS derivatives include monophosphoryl lipid A mimics, such as aminoalkyl glucosaminide phosphate derivatives e.g. RC-529 [220,221].

Lipid A derivatives include derivatives of lipid A from Escherichia coli such as OM-174. OM-174 is described for example in refs. 222 & 223.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 224, 225 and 226 disclose possible analog substitutions e.g. replacement of guanosine with 2′-deoxy-7-deazaguanosine. The adjuvant effect of CpG oligonucleotides is further discussed in refs. 227-232.

The CpG sequence may be directed to TLR9, such as the motif GTCGTT or TTCGTT [233]. The CpG sequence may be specific for inducing a Th1 immune response, such as a CpG-A ODN, or it may be more specific for inducing a B cell response, such a CpG-B ODN. CpG-A and CpG-B ODNs are discussed in refs. 234-236. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 233 & 237-239.

Other immunostimulatory oligonucleotides include a double-stranded RNA, or an oligonucleotide containing a palindromic sequence, or an oligonucleotide containing a poly(dG) sequence.

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 240 and as parenteral adjuvants in ref. 241. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 242-249. Numerical reference for amino acid substitutions is preferably based on the alignments of the A and B subunits of ADP-ribosylating toxins set forth in ref. 250, specifically incorporated herein by reference in its entirety.

Compounds of formula I, II or III, or salts thereof, can also be used as adjuvants:

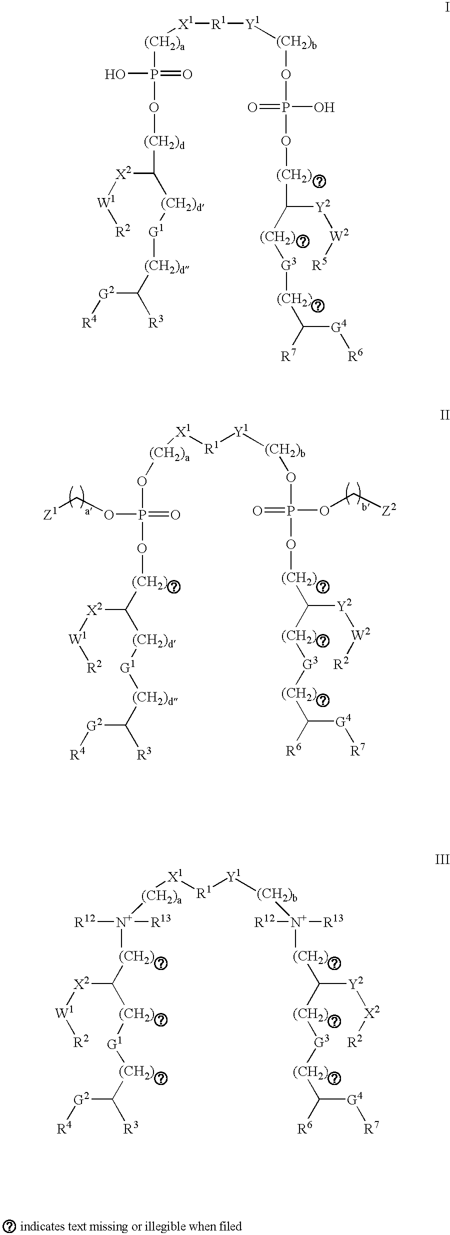

as defined in reference 251, such as ‘ER 803058’, ‘ER 803732’, ‘ER 804053’, ER 804058’, ‘ER 804059’, ‘ER 804442’, ‘ER 804680’, ‘ER 804764’, ER 803022 or ‘ER 804057’ e.g.:

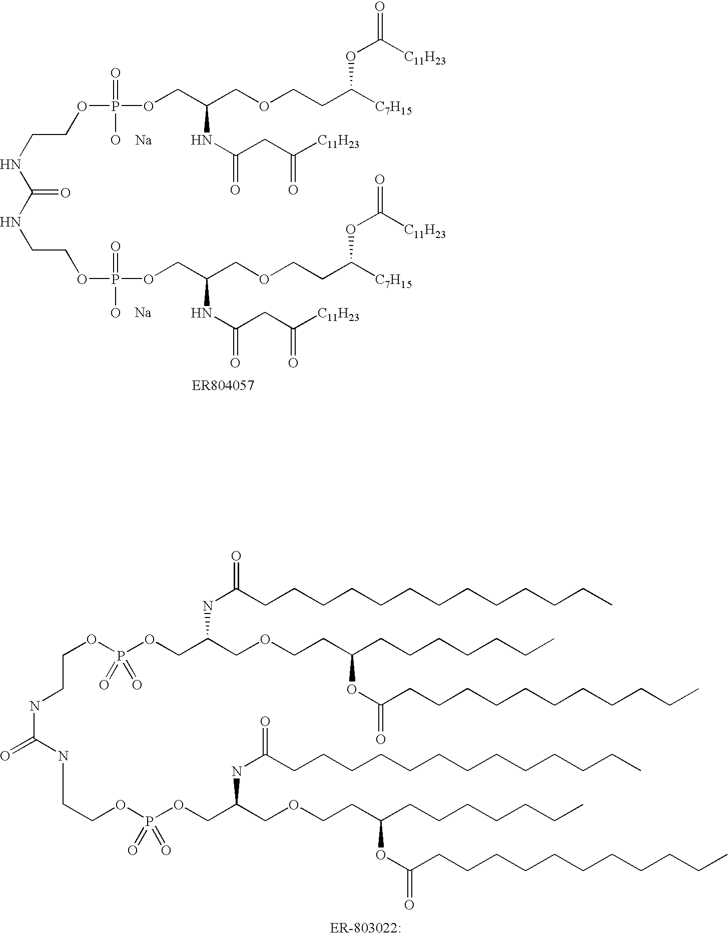

F. Human Immunomodulators

Human immunomodulators suitable for use as adjuvants in the invention include cytokines, such as interleukins (e.g. IL-1, IL-2, IL-4, IL-5, IL-6, IL-7, IL-12 [252], IL-23, IL27 [253] etc.) [254], interferons (e.g. interferon-γ), macrophage colony stimulating factor, tumor necrosis factor and macrophage inflammatory protein-I alpha (MIP-1alpha) and MIP-1beta [255].

G. Bioadhesives and Mucoadhesives

Bioadhesives and mucoadhesives may also be used as adjuvants in the invention. Suitable bioadhesives include esterified hyaluronic acid microspheres [256] or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention [257].

H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

I. Liposomes (Chapters 13 & 14 of Ref 197)

Examples of liposome formulations suitable for use as adjuvants are described in refs. 258-260.

J. Polyoxyethylene Ether and Polyoxyethylene Ester Formulations

Adjuvants suitable for use in the invention include polyoxyethylene ethers and polyoxyethylene esters [261]. Such formulations further include polyoxyethylene sorbitan ester surfactants in combination with an octoxynol [262] as well as polyoxyethylene alkyl ethers or ester surfactants in combination with at least one additional non-ionic surfactant such as an octoxynol [263]. Preferred polyoxyethylene ethers are selected from the following group: polyoxyethylene-9-lauryl ether (laureth 9), polyoxyethylene-9-steoryl ether, polyoxyethylene-8-steoryl ether, polyoxyethylene-4-lauryl ether, polyoxyethylene-3 5-lauryl ether, and polyoxyethylene-23-lauryl ether.

K. Phosphazenes (e.g. PCPP)

Phosphazene adjuvants include poly[di(carboxylatophenoxy)phosphazene] (“PCPP”) as described, for example, in references 264 and 265.

L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

M. Imidazoquinolines

Imidazoquinoline adjuvants include Imiquimod (“R-837”) [266,267], Resiquimod (“R-848”) [268], and their analogs; and salts thereof (e.g. the hydrochloride salts). Further details about immunostimulatory imidazoquinolines can be found in references 269 to 273.

N. Thiosemicarbazones

Thiosemicarbazone adjuvants include those disclosed in reference 274. Methods of formulating, manufacturing, and screening for active compounds are also described in reference 274. The thiosemicarbazones are particularly effective in the stimulation of human peripheral blood mononuclear cells for the production of cytokines, such as TNF-α.

O. Tryptanthrins

Tryptanthrin adjuvants include those disclosed in reference 275. Methods of formulating, manufacturing, and screening for active compounds are also described in reference 275. The thiosemicarbazones are particularly effective in the stimulation of human peripheral blood mononuclear cells for the production of cytokines, such as TNF-α.

P. Nucleoside Analogs

Various nucleoside analogs can be used as adjuvants, such as (a) Isatorabine (ANA-245; 7-thia-8-oxoguanosine):

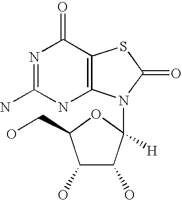

and prodrugs thereof; (b) ANA975; (c) ANA-025-1; (d) ANA380; (e) the compounds disclosed in references 276 to 278; (f) a compound having the formula:

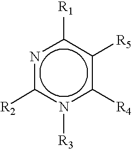

wherein:
R₁ and R₂ are each independently H, halo, —NRₐR_{b}, —OH, C₁₋₆ alkoxy, substituted C₁₋₆ alkoxy, heterocyclyl, substituted heterocyclyl, C₆₋₁₀ aryl, substituted C₆₋₁₀ aryl, C₁₋₆ alkyl, or substituted C₁₋₆ alkyl;
R₃ is absent, H, C₁₋₆ alkyl, substituted C₁₋₆ alkyl, C₆₋₁₀ aryl, substituted C₆₋₁₀ aryl, heterocyclyl, or substituted heterocyclyl;
R₄ and R₅ are each independently H, halo, heterocyclyl, substituted heterocyclyl, —C(O)—R_{d}, C₁₋₆ alkyl, substituted C₁₋₆ alkyl, or bound together to form a 5 membered ring as in R₄₋₅:

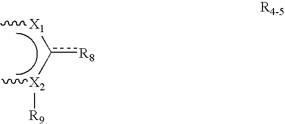

the binding being achieved at the bonds indicated by [symbol]
X₁ and X₂ are each independently N, C, O, or S;
R₈ is H, halo, —OH, C₁₋₆ alkyl, C₂₋₆ alkenyl, C₂₋₆ alkynyl, —OH, —NRₐR_{b}, —(CH₂)ₙ—O—R_{c}, —O—(C₁₋₆ alkyl), —S(O)ₚRₑ, or —C(O)—R_{d};
R₉ is H, C₁₋₆ alkyl, substituted C₁₋₆ alkyl, heterocyclyl, substituted heterocyclyl or R₉ₐ, wherein R₉ₐ is:

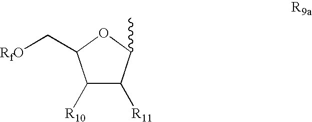

the binding being achieved at the bond indicated by a [symbol]
R₁₀ and R₁₁ are each independently H, halo, C₁₋₆ alkoxy, substituted C₁₋₆ alkoxy, —NRₐR_{b}, or —OH;
each Rₐ and R_{b} is independently H, C₁₋₆ alkyl, substituted C₁₋₆ alkyl, —C(O)R_{d}, C₆₋₁₀ aryl;
each R_{c} is independently H, phosphate, diphosphate, triphosphate, C₁₋₆ alkyl, or substituted C₁₋₆ alkyl;
each R_{d} is independently H, halo, C₁₋₆ alkyl, substituted C₁₋₆ alkyl, C₁₋₆ alkoxy, substituted C₁₋₆ alkoxy, —NH₂, —NH(C₁₋₆ alkyl), —NH(substituted C₁₋₆ alkyl), —N(C₁₋₆ alkyl)₂, —N(substituted C₁₋₆ alkyl)₂, C₆₋₁₀ aryl, or heterocyclyl;
each Rₑ is independently H, C₁₋₆ alkyl, substituted C₁₋₆ alkyl, C₆₋₁₀ aryl, substituted C₆₋₁₀ aryl, heterocyclyl, or substituted heterocyclyl;
each R_{f} is independently H, C₁₋₆ alkyl, substituted C₁₋₆ alkyl, —C(O)R_{d}, phosphate, diphosphate, or triphosphate;
each n is independently 0, 1, 2, or 3;
each p is independently 0, 1, or 2; or
   or (g) a pharmaceutically acceptable salt of any of (a) to (f), a tautomer of any of (a) to (f), or a pharmaceutically acceptable salt of the tautomer.

Q. Lipids Linked to a Phosphate-Containing Acyclic Backbone

Adjuvants containing lipids linked to a phosphate-containing acyclic backbone include the TLR4 antagonist E5564 [279,280]:

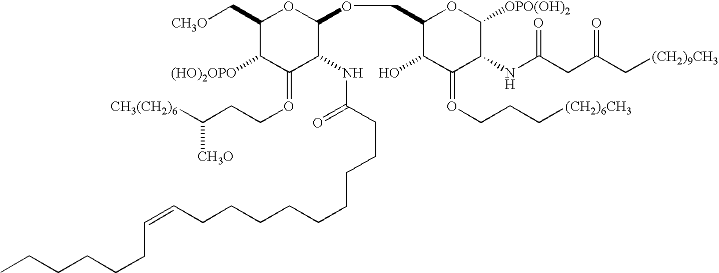

R. Small Molecule Immunopotentiators (SMIPs)

SMIPs include:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine;
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c]quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine;
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine;
2-[[4-amino-1-(2-methylpropyl)-1H-imidazo[4,5-c]quinolin-2-yl](methyl)amino]ethanol;
2-[[4-amino-1-(2-methylpropyl)-1H-imidazo[4,5-c]quinolin-2-yl](methyl)amino]ethyl acetate;
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine;
1-{4-amino-2-[methyl(propyl)amino]-1H-imidazo[4,5-c]quinolin-1-yl}-2-methylpropan-2-ol;
1-[4-amino-2-(propylamino)-1H-imidazo[4,5-c]quinolin-1-yl]-2-methylpropan-2-ol;
N4,N4-dibenzyl-1-(2-methoxy-2-methylpropyl)-N2-propyl-1H-imidazo[4,5-c]quinoline-2,4-diamine.

S. Proteosomes

One adjuvant is an outer membrane protein proteosome preparation prepared from a first Gram-negative bacterium in combination with a liposaccharide preparation derived from a second Gram-negative bacterium, wherein the outer membrane protein proteosome and liposaccharide preparations form a stable non-covalent adjuvant complex. Such complexes include “IVX-908”, a complex comprised of Neisseria meningitidis outer membrane and lipopolysaccharides. They have been used as adjuvants for influenza vaccines [281].

T. Other Adjuvants

Other substances that act as immunostimulating agents are disclosed in references 197 and 202. Further useful adjuvant substances include:
Methyl inosine 5′-monophosphate (“MIMP”) [282].
A polyhydroxlated pyrrolizidine compound [283], such as one having formula:

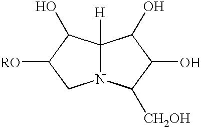

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
A gamma inulin [284] or derivative thereof, such as algammulin.
Compounds disclosed in reference 285.
Compounds disclosed in reference 286, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [287,288], Hydrapthalamide compounds, Benzophenone compounds, Isoxazole compounds, Sterol compounds, Quinazilinone compounds, Pyrrole compounds [289], Anthraquinone compounds, Quinoxaline compounds, Triazine compounds, Pyrazalopyrimidine compounds, and Benzazole compounds [290].
Loxoribine (7-allyl-8-oxoguanosine) [291].
A formulation of a cationic lipid and a (usually neutral) co-lipid, such as aminopropyl-dimethyl-myristoleyloxy-propanaminium bromide-diphytanoylphosphatidyl-ethanolamine (“Vaxfectin™”) or aminopropyl-dimethyl-bis-dodecyloxy-propanaminium bromide-dioleoylphosphatidyl-ethanolamine (“GAP-DLRIE:DOPE”). Formulations containing (±)-N-(3-aminopropyl)-N,N-dimethyl-2,3-bis(syn-9-tetradeceneyloxy)-1-propanaminium salts are preferred [292].

The invention may also comprise combinations of aspects of one or more of the adjuvants identified above. For example, the following adjuvant compositions may be used in the invention: (1) a saponin and an oil-in-water emulsion [293]; (2) a saponin (e.g. QS21)+a non-toxic LPS derivative (e.g. 3dMPL) [294]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [295]; (5) combinations of 3dMPL with, for example, QS21 and/or oil-in-water emulsions [296]; (6) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (7) one or more mineral salts (such as an aluminum salt)+a non-toxic derivative of LPS (such as 3dMPL).

Adjuvants used in addition to GPs in the present invention may be modulators and/or agonists of Toll-Like Receptors (TLR). For example, they may be agonists of one or more of the human TLR1, TLR2, TLR3, TLR4, TLR7, TLR8, and/or TLR9 proteins. Preferred agents are agonists of TLR7 (e.g. imidazoquinolines) and/or TLR9 (e.g. CpG oligonucleotides). These agents are useful for activating innate immunity pathways.

General

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### BRIEF DESCRIPTION OF DRAWINGS

FIG. 1: x axis shows the mice groupings (intranasal immunization with TT alone (2.5 μg) or with TT+GLP-1(19 μg) or with TT+LT (E. coli heat-labile enterotoxin, 1 μg) and the y axis shows the anti-TT serum IgG (geometric mean titer) in serum taken one week after the fourth dose.

FIG. 2: x axis shows the mice groupings (mice 2-4 were intranasally immunized four times with Tetanus Toxoid (TT, 2.5 micrograms) alone, mice 5-8 were intranasally immunized with TT+GLP-1 at 19 micrograms). The y axis shows the anti TT IgA serum titer one week after the fourth dose.

FIG. 3: structure of the mammalian preproglucagon product. GRPP=glicentin-related pancreatic peptide. IP=intervening peptide. GLP-2=glucagon-related peptide-2. Additional peptides are derived from the preproprotein including: glicentin which is composed of amino acids 1-69, oxyntomodulin (amino acids 30-69) and the major proglucagon fragment (MPGF) (amino acids 72-158).

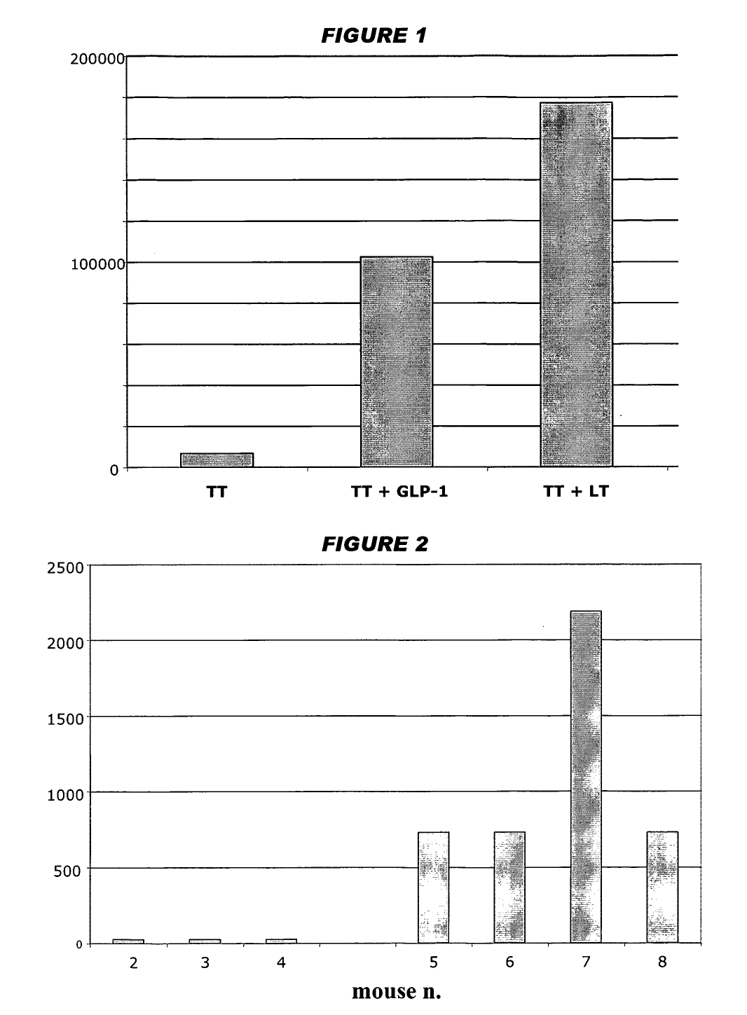

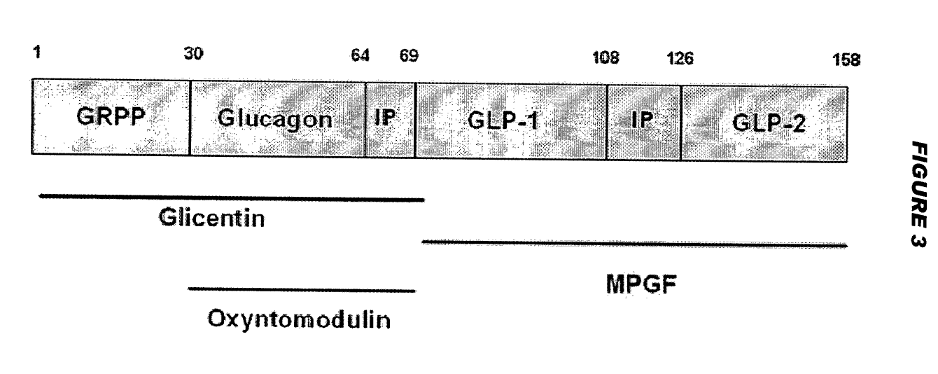

### BRIEF DESCRIPTION OF THE SEQUENCE LISTING

Sequence 1—GLP-1 amino acid sequence

Sequence 2—GLP-2 amino acid sequence

Sequence 3—Glucagon amino acid sequence

Sequence 4—Secretin amino acid sequence

Sequence 5—VIP amino acid sequence

Sequence 6—GIP amino acid sequence

Sequence 7—PACAP (1-38) amino acid sequence

Sequence 8—Gastrin amino acid sequence

Sequence 9—Cholecystokinin preproprotein amino acid sequence

Sequence 10—Motilin amino acid sequence

Sequence 11—Pancreatic peptide amino acid sequence

### MODES FOR CARRYING OUT THE INVENTION

Groups of four mice (C57/BL6; 6-8 weeks; 20-25 g) were intranasally immunised four times at weekly intervals (days 0, 7, 14, 21) with 2.5 μg of tetanus toxoid (TT) in the absence or in the presence of 19 μg GLP-1 or 1 μg of E. coli heat-labile enterotoxin (LT). For intranasal immunisation, mice were lightly anaesthetised by intraperitoneal injection of ketamine and xylazine and a final volume of 15-20 μl of a solution containing antigen with or without adjuvant was administered (7.5-10 μl per nostril). Serum samples were collected every week, 24 hr before each immunisation and one week after the last dose and were stored at −20° C. until assayed.

Anti-TT antibodies were titrated in individual serum samples by using ELISA methods. Microplates (Microtest III, Becton Dickinson) were coated with a 100 μl solution of TT (5 μg/ml) in PBS and incubated overnight at 4° C. Plates were washed three times with PBS containing 0.05% Tween-20, blocked for 2 hours with 200 μl of PBS containing 1% BSA and serial dilutions of serum or samples were added to duplicate wells. IgG and IgA were determined by addition of gamma-chain-specific or alpha-chain specific biotin-conjugated goat anti-mouse antibodies diluted 1:1000 in PBS containing 0.1% BSA and 0.025% Tween-20. After incubation and washing steps, a 100 μl aliquot of HRP-conjugated streptavidin (Dako, Glostrup, Denmark) diluted 1:2000 in PBS containing 0.1% BSA and 0.025% Tween-20, will be added and colour developed with 3,3′,5,5′-tetramethylbenzidine (TMB) substrate. The color reaction was terminated after 5-10 min. with 50 μl of 0.2 M H₂SO₄ and absorbance at 450 nm was determined with an ELISA plate reader. Antibody titers are expressed as the reciprocal of the sample dilution corresponding to an optical density of 0.3 units (for IgG) or 0.2 units (for IgA) above controls. Antibody titers in each group of mice are reported as geometric mean of individually measured titers (GMT).

The mice vaccinated with TT+GLP-1 developed high anti-TT serum IgG titers as compared to mice vaccinated with TT only (geometric mean titer 102,276 vs. 6,561). The IgG response of the mice that received TT+LT was 177,147. The serum IgA response was 27 (GMT of three mice) in mice that received TT alone and 959 (GMT of four mice) in mice immunised with GLP-1.

Therefore, GLP-1 is an effective mucosal adjuvant.

In a further assay, mice (C57/BL6) received four weekly doses of TT (2 μg/dose) with or without GLP-1 (30 μg/dose). Two months later the mice were challenged subcutaneously with DP50 (50 times the dose paralyzing 50% of the animals, as established in preliminary experiments) of tetanus toxin and paralysis and death were monitored for one week. The mice immunized with TT alone were not protected (0/7 survivors) whereas the mice that received the antigen with the adjuvant GLP-1 were all protected (8/8 survivors and mice showed no sign of paralysis). Furthermore, the serum IgG titers specific for the antigen were analyzed immediately before the challenge. The range of anti-TT IgG titer for mice immunized with TT alone was 256-8,192 as compared with 16,384-131,072 for mice that received the antigen with the adjuvant GLP-1.

These results demonstrate that (1) GLP-1 induces protective responses to the co-administered antigen; (2) mucosal (intranasal) immunization with GLP-1 induces protective responses against a systemic (subcutaneous) challenge; and (3) GLP-1 induces “memory” protective responses as the challenge was performed two months after the last vaccination dose. The anti-TT serum IgG titers after two months were high and two months is a significant amount of time for the mouse lifespan.

It will be understood that the invention has been described by way of example only and modifications may be made whilst remaining within the scope and spirit of the invention.

### REFERENCES (THE CONTENTS OF WHICH ARE HEREBY INCORPORATED IN FULL)

[1] Drucker D J. (1998) Diabetes. February;47(2):159-69.

[2] Drucker D J. (2001) Curr Pharm Des. September;7(14):1399-412.

[3] Orskov C. et al., (1994) Diabetes. April;43(4):535-9.

[4] Velazquez E et al., (2003) Eur J Biochem. July;270(14):3001-9.

[5] Drucker D J et al., (1997) Nat Biotechnol. July;15(7):673-7.

[6] Blache P et al., (1993) J Biol Chem. Oct. 15;268(29):21748-53.

[7] Couvineau A et al., (1984) Biochem Biophys Res Commun. Jun. 15;121(2):493-8.

[8] Thorens B et al. (1993) Diabetes 1993; 42(11): 1678-82.

[9] Uchida D et al., (1998) Ann N Y Acad Sci. Dec. 11;865:253-8

[10] Ogawa & Uchida (1995) FEMS Immunol. Med. Microbiol. 11:197-205.

[11]Vaccines, 4th Edition (Plotkin and Orenstein ed.) 2004

[12] Del Giudice et al. (1998) Molecular Aspects of Medicine 19:1-70.

[13] Medical Microbiology 4th Edition (Murray et al. ed. 2002)

[14] Virology, 3rd Edition (W. K. Joklik ed. 1988)

[15] Fundamental Virology, 2nd Edition (B. N. Fields and D. M. Knipe, eds. 1991)

[16] WO99/24578

[17] WO99/36544

[18] WO99/57280

[19] WO00/22430.

[20] Tettelin et al. (2000) Science 287:1809-1815

[21] WO96/29412

[22] Pizza et al. (2000) Science 287:1816-1820

[23] WO01/52885

[24] Bjune et al. (1991) Lancet 338(8775):1093-96

[25] Fukasawa et al. (1999) Vaccine 17:2951-2958.

[26] Rosenqvist et al. (1998) Dev. Biol. Stand. 92:323-333.

[27] Costantino et al. (1992) Vaccine 10:691-698.

[28] Costantino et al. (1999) Vaccine 17:1251-1263.

[29] WO03/007985.

[30] WO00/66791

[31] WO01/64922

[32] WO01/64920

[33] WO03/020756

[34] WO2004/032958

[35] WO2004/048404.

[36] WO98/18931

[37] WO98/18930

[38] U.S. Pat. No. 6,699,703

[39] U.S. Pat. No. 6,800,744

[40] WO97/43303

[41] WO97/37026

[42] Watson (2000) Pediatr Infect Dis J 19:331-332.

[43] Rubin (2000) Pediatr Clin North Am 47:269-285, v.

[44] Jedrzejas (2001) Microbiol Mol Biol Rev 65:187-207.

[45] WO02/22167.

[46] Paoletti et al., (1990) J Biol Chem 265:18278-83.

[47] Wessels et al., (1990) J Clin Invest 86:1428-33.

[48] Baker et al., (2004) J Infect Dis 171:879-84.

[49] WO02/34771

[50] WO2005/032582

[51] WO02/094851

[52] Dale, Vaccine (1999) 17:193-200

[53] Dale, Vaccine 14(10): 944-948

[54] Dale (1999) Infect Dis Clin North Am 13:227-43, viii.

[55] Ferretti et al. (2001) PNAS USA 98: 4658-4663.

[56] WO02/18595

[57] WO99/58562

[58] McMichael (2000) Vaccine 19 Suppl 1:S101-107.

[59] Gustafsson et al. (1996) N. Engl. J. Med. 334:349-355.

[60] Rappuoli et al. (1991) TIBTECH 9:232-238.

[61] Kuroda et al. (2001) Lancet 357(9264):1225-1240; see also pages 1218-1219.

[62] WO00/56360

[63] Infect Immun. 2001 May; 69(5): 3510-3515

[64] WO03/093306

[65] Schuchat (1999) Lancer 353(9146):51-6

[66] WO2004/041157

[67] WO2005/002619

[68] Zhu et al., Vaccine (2004) 22:660-669

[69] Price et al., Infection and Immunity (2004) 71(1):277-283)

[70] Plante et al., J Infectious Disease (2000)182:848-855)

[71] WO02/079243

[72] WO00/37494

[73] WO03/049762

[74] WO03/068811

[75] WO99/28475.

[76] U.S. Pat. No. 6,756,361

[77] Covacci & Rappuoli (2000) J. Exp. Med. 19:587-592.

[78] WO93/18150.

[79] WO99/53310

[80] Covacci et al. (1993) Proc. Natl. Acad. Sci. USA 90: 5791-5795.

[81] Tummuru et al. (1994) Infect. Immun. 61:1799-1809.

[82] Marchetti et al. (1998) Vaccine 16:33-37.

[83] Telford et al. (1994) J. Exp. Med. 179:1653-1658.

[84] Evans et al. (1995) Gene 153:123-127.

[85] WO96/01272 & WO96/01273, especially SEQ ID NO:6.

[86] WO97/25429.

[87] WO98/04702.

[88] Infect Immun. 2002 August; 70(8):4414

[89] J Toxicol Clin Toxicol (2001) 39:85-100.

[90] Demicheli et al. (1998) Vaccine 16:880-884.

[91] Stepanov et al. (1996) J Biotechnol 44:155-160.

[92] Infect Immun. 2003 January; 71(1):374-383.

[93] Infect Immun. 1999 October; 67(10):5395

[94] Infect Immun. 1997 November; 65(11):4476-4482

[95] Infect Immun. 2004 October; 72(10): 6148

[96] Proc Natl Acad Sci USA 2004 Aug. 24; 101(34):12652

[97] Infect Immun. 2004 July; 72(7):3829

[98] Biochim Biophys Acta. 2004 Nov. 1;1702(2):145

[99] J Autoimmun. 1989 June; 2 Suppl:81

[100] WO02/02606

[101] Kalman et al. (1999) Nature Genetics 21:385-389.

[102] Read et al. (2000) Nucleic Acids Res 28:1397-406.

[103] Shirai et al. (2000) J. Infect. Dis. 181(Suppl 3):S524-S527.

[104] WO99/27105.

[105] WO00/27994.

[106] Infect Immun. 2003 Oct. 7; 71(10):5498-504

[107] Infect Immun. 2001 May; 69(5):3323-3334

[108] J Clin Microbiol. 1999 December; 37(12):3997

[109] Ross et al. (2001) Vaccine 19:4135-4142.

[110] U.S. Pat. No. 5,360,897

[111] Can J Biochem Cell Biol. 1984 May; 62(5):270-5

[112] Anderson (2000) Vaccine 19 Suppl 1:S59-65.

[113] Kahn (2000) Curr Opin Pediatr 12:257-262.

[114] Crowe (1995) Vaccine 13:415-421.

[115] J Gen Virol. 2004 November; 85(Pt 11):3229

[116] Sutter et al. (2000) Pediatr Clin North Am 47:287-308.

[117] Zimmerman & Spann (1999) Am Fam Physician 59:113-118, 125-126.

[118] Bell (2000) Pediatr Infect Dis J 19:1187-1188.

[119] Iwarson (1995) APMIS 103:321-326.

[120] Gerlich et al. (1990) Vaccine 8 Suppl:S63-68 & 79-80.

[121] Houghton et al., Hepatology (1991) 14:381

[122] Hsu et al (1999) Clin Liver Dis 3:901-915.

[123] Dreesen (1997) Vaccine 15 Suppl:S2-6.

[124] MMWR Morb Mortal Wkly Rep 1998 Jan. 16;47(1):12, 19.

[125] WO2004/92360

[126] U.S. Pat. No. 5,378,814

[127] U.S. Pat. No. 6,333,164

[128] WO00/15255

[129] US Patent Application 20020007173.

[130] U.S. Pat. No. 5,693,522.

[131] Moingeon (2001) Vaccine 19:1305-1326.

[132] Rosenberg (2001) Nature 411:380-384.

[133] Dermine, S. et al, “Cancer Vaccines and Immunotherapy,” British Medical Bulletin, 2002, 62, 149-162

[134] Espinoza-Delgado I., “Cancer Vaccines,” The Oncologist, 2002, 7(suppl 3):20-33

[135] Davis, I. D. et al., “Rational approaches to human cancer immunotherapy,” Journal of Leukocyte Biology, 2003, 23:3-29

[136] Van den Eynde B, et al., “New tumor antigens recognized by T cells”, Curr. Opin. Immunol., 1995, 7:674-81

[137] Rosenberg S A, “Cancer vaccines based on the identification of genes encoding cancer regression antigens, Immunol. Today, 1997, 18:175-82

[138] Offringa R et al., “Design and evaluation of antigen-specific vaccination strategies against cancer”, Current Opin. Immunol., 2000, 2:576-582

[139] Rosenberg S A, “A new era for cancer immunotherapy based on the genes that encode cancer antigens,” Immunity, 1999, 10:281-7

[140] Sahin U et al., “Serological identification of human tumor antigens,” Curr. Opin. Immunol., 1997, 9:709-16

[141] Old L J et al., “New paths in human cancer serology,” J. Exp. Med., 1998, 187:1163-7

[142] Chaux et al., “Identification of MAGE-3 epitopes presented by HLA-DR molecules to CD4(+) T lymphocytes,” J. Exp. Med., 1999, 189:767-78

[143] Gold P, et al., “Specific carcinoembryonic antigens of the human digestive system,” J. Exp. Med., 1965, 122:467-8

[144] Livingston P O, et al., Carbohydrate vaccines that induce antibodies against cancer: Rationale,” Cancer Immunol. Immunother., 1997, 45:1-6

[145] Livingston P O, et al., Carbohydrate vaccines that induce antibodies against cancer: Previous experience and future plans,” Cancer Immunol. Immunother., 1997, 45:10-9

[146] Taylor-Papadimitriou J, “Biology, biochemistry and immunology of carcinoma-associated mucins,” Immunol. Today, 1997, 18:105-7

[147] Zhao X -J et al., “GD2 oligosaccharide: target for cytotoxic T lymphocytes,” J. Exp. Med., 1995, 182:67-74

[148] Theobald M, et al., “Targeting p53 as a general tumor antigen,” Proc. Natl. Acad. Sci. USA, 1995, 92:11993-7

[149] Gaudernack G, “T cell responses against mutant ras: a basis for novel cancer vaccines,” Immunotechnology, 1996, 2:3-9

[150] WO91/02062

[151] U.S. Pat. No. 6,015,567

[152] WO01/08636

[153] WO96/30514

[154] U.S. Pat. No. 5,846,538

[155] U.S. Pat. No. 5,869,445

[156] Ingram (2001) Trends Neurosci 24:305-307.

[157] U.S. Pat. No. 6,884,435.

[158] Ramsay et al. (2001) Lancet 357(9251):195-196.

[159] Lindberg (1999) Vaccine 17 Suppl 2:S28-36.

[160] Buttery & Moxon (2000) J R Coll Physicians Lond 34:163-168.

[161] Ahmad & Chapnick (1999) Infect Dis Clin North Am 13:113-133, vii.

[162] Goldblatt (1998) J. Med. Microbiol. 47:563-567.

[163] EP-A-0477508.

[164] U.S. Pat. No. 5,306,492.

[165] WO98/42721.

[166] Wong (1991) Chemistry of Protein Conjugation and Cross-Linking. ISBN 0-8493-5886-8.

[167] Conjugate Vaccines (eds. Cruse et al.) Karger, Basel, 1989, partic. Vol. 10, pp. 48-114.

[168] Hermanson Bioconjugate Techniques, Academic Press, San Diego (1996) ISBN: 0123423368.

[169] Anonymous (January 2002) Research Disclosure, 453077.

[170] Anderson (1983) Infect Immun 39(1):233-238.

[171] Anderson et al. (1985) J Clin Invest 76(1):52-59.

[172] EP-A-0372501.

[173] EP-A-0378881.

[174] EP-A-0427347.

[175] WO93/17712

[176] WO94/03208.

[177] WO98/58668.

[178] EP-A-0471177.

[179] WO91/01146

[180] Falugi et al. (2001) Eur J Immunol 31:3816-3824.

[181] Baraldo et al, (2004) Infect Immun. 72:4884-7

[182] EP-A-0594610.

[183] WO02/091998.

[184] Kuo et al. (1995) Infect Immun 63:2706-13.

[185] WO01/72337

[186] WO00/61761.

[187] Charalambous & Feavers (2001) J Med Microbiol 50:937-939.

[188] Molecular Cloning: A Laboratory Manual (Third Edition) Cold Spring Harbour Laboratory Press

[189] 1998, eds. “Gene expression systems. Using nature for the art of expression”. Academic Press, San Diego, London, Boston, New York, Sydney, Tokyo, Toronto

[190] Gennaro (2000) Remington: The Science and Practice of Pharmacy. 20th edition, ISBN: 0683306472.

[191] Almeida & Alpar (1996) J. Drug Targeting 3:455-467.

[192] Agarwal & Mishra (1999) Indian J Exp Biol 37:6-16

[193] Walker (1994) Vaccine 12:387-400

[194] Clements (1997) Nature Biotech. 15:622-623

[195] McGhee et al. (1992) Vaccine 10:75-88

[196] Michetti (1998) J. Gastroenterol. [Suppl X]:66-68

[197] Vaccine Design . . . (1995) eds. Powell & Newman. ISBN: 030644867X. Plenum.

[198] WO00/23105.

[199] WO90/14837.

[200] Podda & Del Giudice (2003) Expert Rev Vaccines 2:197-203.

[201] Podda (2001) Vaccine 19: 2673-2680.

[202] Vaccine Adjuvants: Preparation Methods and Research Protocols (Volume 42 of Methods in Molecular Medicine series). ISBN: 1-59259-083-7. Ed. O'Hagan.

[203] Allison & Byars (1992) Res Immunol 143:519-25.

[204] Hariharan et al. (1995) Cancer Res 55:3486-9.

[205] U.S. Pat. No. 5,057,540.

[206] WO96/33739.

[207] EP-A-0109942.

[208] WO96/11711.

[209] WO00/07621.

[210] Barr et al. (1998) Advanced Drug Delivery Reviews 32:247-271.

[211] Sjolanderet et al. (1998) Advanced Drug Delivery Reviews 32:321-338.

[212] Niikura et al. (2002) Virology 293:273-280.

[213] Lenz et al. (2001) J Immunol 166:5346-5355.

[214] Pinto et al. (2003) J Infect Dis 188:327-338.

[215] Gerber et al. (2001) Virol 75:4752-4760.

[216] WO03/024480

[217] WO03/024481

[218] Gluck et al. (2002) Vaccine 20:B10-B16.

[219] EP-A-0689454.

[220] Johnson et al. (1999) Bioorg Med Chem Lett 9:2273-2278.

[221] Evans et al. (2003) Expert Rev Vaccines 2:219-229.

[222] Meraldi et al. (2003) Vaccine 21:2485-2491.

[223] Pajak et al. (2003) Vaccine 21:836-842.

[224] Kandimalla et al (2003) Nucleic Acids Research 31:2393-2400.

[225] WO02/26757.

[226] WO99/62923.

[227] Krieg (2003) Nature Medicine 9:831-835.

[228] McCluskie et al. (2002) FEMS Immunology and Medical Microbiology 32:179-185.

[229] WO98/40100.

[230] U.S. Pat. No. 6,207,646.

[231] U.S. Pat. No. 6,239,116.

[232] U.S. Pat. No. 6,429,199.

[233] Kandimalla et al. (2003) Biochemical Society Transactions 31 (part 3):654-658.

[234] Blackwell et al. (2003) J Immunol 170:4061-4068.

[235] Krieg (2002) Trends Immunol 23:64-65.

[236] WO01/95935.

[237] Kandimalla et al. (2003) BBRC 306:948-953.

[238] Bhagat et al. (2003) BBRC 300:853-861.

[239] WO03/035836.

[240] WO95/17211.

[241] WO98/42375.

[242] Beignon et al. (2002) Infect Immun 70:3012-3019.

[243] Pizza et al. (2001) Vaccine 19:2534-2541.

[244] Pizza et al. (2000) Int J Med Microbiol 290:455-461.

[245] Scharton-Kersten et al. (2000) Infect Immun 68:5306-5313.

[246] Ryan et al. (1999) Infect Immun 67:6270-6280.

[247] Partidos et al. (1999) Immunol Lett 67:209-216.

[248] Peppoloni et al. (2003) Expert Rev Vaccines 2:285-293.

[249] Pine et al. (2002) J Control Release 85:263-270.

[250] Domenighini et al. (1995) Mol Microbiol 15:1165-1167.

[251] WO03/011223.

[252] WO99/40936.

[253] Matsui M. et al. (2004) J. Virol 78: 9093.

[254] WO99/44636.

[255] Lillard J W et al., (2003) Blood Feb. 1;101(3):807-14. Epub 2002 Sep. 12.

[256] Singh et al] (2001) J Cont Release 70:267-276.

[257] WO99/27960.

[258] U.S. Pat. No. 6,090,406

[259] U.S. Pat. No. 5,916,588

[260] EP-A-0626169.

[261] WO99/52549.

[262] WO01/21207.

[263] WO01/21152.

[264] Andrianov et al. (1998) Biomaterials 19:109-115.

[265] Payne et al. (1998) Adv Drug Delivery Review 31:185-196.

[266] U.S. Pat. No. 4,680,338.

[267] U.S. Pat. No. 4,988,815.

[268] WO92/15582.

[269] Stanley (2002) Clin Exp Dermatol 27:571-577.

[270] Wu et al. (2004) Antiviral Res. 64(2):79-83.

[271] Vasilakos et al. (2000) Cell Immunol. 204(1):64-74.

[272] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.

[273] Jones (2003) Curr Opin Investig Drugs 4:214-218.

[274] WO2004/060308.

[275] WO2004/064759.

[276] U.S. Pat. No. 6,924,271.

[277] US2005/0070556.

[278] U.S. Pat. No. 5,658,731.

[279] Wong et al. (2003) J Clin Pharmacol 43(7):735-42.

[280] US2005/0215517.

[281] WO02/072012.

[282] Signorelli & Hadden (2003) Int Immunopharmacol 3(8):1177-86.

[283] WO2004/064715.

[284] Cooper (1995) Pharm Biotechnol 6:559-80.

[285] PCT/US2005/022769.

[286] WO2004/87153.

[287] U.S. Pat. No. 6,605,617.

[288] WO02/18383.

[289] WO2004/018455.

[290] WO03/082272.

[291] U.S. Pat. No. 5,011,828.

[292] U.S. Pat. No. 6,586,409.

[293] WO99/11241.

[294] WO94/00153.

[295] WO98/57659.

[296] European patent applications 0835318, 0735898 and 0761231.

1. An immunogenic composition comprising (a) a gastrointestinal peptide adjuvant; and (b) at least one antigen.

2. The composition of claim 1 wherein the gastrointestinal peptide adjuvant is a mucosal adjuvant.

3. The composition of claim 1, wherein the gastrointestinal peptide adjuvant is a molecule which is secreted by enteroendocrine cells and by gastrointestinal nerves.

4. The composition of claim 3, wherein the gastrointestinal peptide is a molecule which can bind to G protein coupled receptors.

5. The composition of claim 4, wherein the gastrointestinal peptide is a molecule which can bind to Gs protein coupled receptors.

6. The immunogenic composition of claim 1, wherein the gastrointestinal peptide is a molecule which can induce cAMP production in epithelial cells.

7. The composition of claim 1, wherein the gastrointestinal peptide is selected from the group consisting of VIP, PACAP, gastrin, cholecystokinin, motilin, neurotensin, secretin, glucagon, miniglucagon, GIP, enteroglucagon, pancreatic polypeptide, glicentin, glicentin-related pancreatic peptide, oxyntomodulin, exendin-4, maxadilan, GLP-1 and GLP-2

8. The composition of claim 7, wherein the gastrointestinal peptide is GLP-1.

9. A method of raising an immune response in a patient against an antigen, comprising the step of administering at least one antigen to a patient in combination with a gastrointestinal peptide adjuvant.

10. The method according to claim 9 wherein the gastrointestinal peptide adjuvant and the antigen are administered simultaneously, sequentially or separately.

11. (canceled)

12. The method of claim 9, wherein the patient is a human.

13. A kit comprising: (a) a gastrointestinal peptide adjuvant and (b) at least one antigen.
